(19) United States
(12) Patent Application Publication
(10) Pub. No.: US 2023/0317267 A1
(43) Pub. Date: Oct. 5, 2023

(54) MEDICAL SERVICE SUPPORT DEVICE, MEDICAL SERVICE SUPPORT METHOD, AND PROGRAM

(71) Applicant: FUJIFILM Corporation, Tokyo (JP)

(72) Inventors: Yosuke Ohashi, Tokyo (JP); Shinichi Komiyama, Tokyo (JP); Chieko Iwanaga, Tokyo (JP); Yuma Hori, Tokyo (JP); Tatsuya Kobayashi, Tokyo (JP)

(73) Assignee: FUJIFILM Corporation, Tokyo (JP)

(21) Appl. No.: 18/332,758

(22) Filed: Jun. 12, 2023

Related U.S. Application Data
(63) Continuation of application No. PCT/JP2021/041783, filed on Nov. 12, 2021.

(30) Foreign Application Priority Data
Dec. 14, 2020 (JP) 2020-206813

(51) Int. Cl. G16H 40/40; A61B 1/12

(52) U.S. Cl. CPC G16H 40/40; A61B 1/123; G16H 40/20

(57) ABSTRACT

The medical service support device includes a processor and a memory connected to or built into the processor. The processor is configured to acquire a washing wait time from when a medical device procedure service using a medical accessory device that is attachably and detachably connected to a medical management device is ended to when washing of the medical accessory device by a washer is started, for each default unit capable of classifying the washing wait time, and output medical service support information in which the washing wait time and the default unit are associated with each other.

### CROSS-REFERENCE TO RELATED APPLICATIONS

This application is a continuation application of International Application No. PCT/JP2021/041783, filed Nov. 12, 2021, the disclosure of which is incorporated herein by reference in its entirety. Further, this application claims priority under 35 USC 119 from Japanese Patent Application No. 2020-206813 filed Dec. 14, 2020, the disclosure of which is incorporated by reference herein.

### BACKGROUND

### 1. Technical Field

The present invention relates to a medical service support device, a medical service support method, and a program.

### 2. Related Art

JP2019-005033A discloses an endoscope management system that manages a washing step of an endoscope, the endoscope management system including: an acquisition unit that acquires time point information indicating a start time point of manual washing and an end time point of manual washing; a time derivation unit that derives execution time of the manual washing based on the time point information acquired by the acquisition unit; a determination unit that compares the execution time of the manual washing, which is derived by the time derivation unit, with reference washing time for the manual washing and determines whether or not the manual washing is appropriately performed; and a notification processing unit that provides a predetermined notification in a case where the determination unit determines that the manual washing is not appropriately performed.

Further, in the endoscope management system described in JP2019-005033A, the acquisition unit acquires the time point information indicating the end time point of an examination, the time derivation unit derives washing wait time from the end of the examination to the start of the manual washing based on the time point information acquired by the acquisition unit, and the determination unit compares the washing wait time, which is derived by the time derivation unit, with allowable wait time from the end of the examination to the start of the manual washing and determines whether or not the manual washing is started appropriately.

Further, in the endoscope management system described in JP2019-005033A, the acquisition unit acquires the time point information indicating the end time point of an examination, the time derivation unit derives the washing wait time from the end of the examination to the current item based on the time point information acquired by the acquisition unit, the determination unit compares the washing wait time, which is derived by the time derivation unit, with allowable wait time from the end of the examination to the start of the manual washing and determines whether or not the washing wait time exceeds the allowable wait time, and the notification processing unit provides the predetermined notification in a case where the determination unit determines that the washing wait time exceeds the allowable wait time.

JP2010-039560A discloses a medical device management support device including: an examination schedule storing unit that stores an examination schedule including a start time point and an end time point of each examination; a washing capability storing unit that stores washing capability information that defines washing capability of a washing device that washes an scope used in the examination; a washing schedule generation unit that generates a washing schedule for washing a used scope, which is generated at the end of each examination, with the washing device having washing capability defined based on the washing capability information stored in the washing capability storing unit according to the examination schedule stored in the examination schedule storing unit; and an scope determination unit that determines whether or not there is a shortage of scopes to be used in each examination according to an examination start time point specified through the examination schedule and a washing end time point specified through the washing schedule.

WO2010/047081A discloses an examination management device that acquires an operation status of a washing device in real time, detects the need for changing an examination schedule as early as possible by executing a simulation for predicting whether there are insufficient endoscopes at predetermined time intervals, and changes the examination schedule at an early stage.

Further, the examination management device described in WO2010/047081A includes: a standard time storing unit that stores each standard time of a plurality of steps constituting one examination according to the examination time; a time point acquisition unit that acquires a start time point and an end time point of each step of an examination from at least one device installed in an examination room where the examination is being performed; and a display control unit that displays a first display content indicating a target progress status of the examination based on the standard time corresponding to an examination time point and a second display content indicating an actual progress status based on the start time point and the end time point of each step of the examination acquired by the time point acquisition unit, on a display portion of a terminal device installed in the examination room.

### SUMMARY

One embodiment according to the present disclosed technology provides a medical service support device, a medical service support method, and a program capable of supporting efficient performance of an endoscope handling service.

A medical service support device according to a first aspect of the present disclosed technology comprises: a processor; and a memory that is connected to or built into the processor, in which the processor is configured to: acquire a washing wait time from when an endoscope procedure service using an endoscope is ended to when washing of the endoscope by a washer is started, for each default unit capable of classifying the washing wait time; and output medical service support information in which the washing wait time and the default unit are associated with each other.

A medical service support method according to a second aspect of the present disclosed technology comprises: acquiring a washing wait time from when an endoscope procedure service using an endoscope is ended to when washing of the endoscope by a washer is started, for each default unit capable of classifying the washing wait time; and outputting medical service support information in which the washing wait time and the default unit are associated with each other.

A program according to a third aspect of the present disclosed technology that causes a computer to execute a process comprises: acquiring a washing wait time from when an endoscope procedure service using an endoscope is ended to when washing of the endoscope by a washer is started, for each default unit capable of classifying the washing wait time; and outputting medical service support information in which the washing wait time and the default unit are associated with each other.

### BRIEF DESCRIPTION OF THE DRAWINGS

Exemplary embodiments of the technology of the disclosure will be described in detail based on the following figures, wherein:

FIG. 1 is a conceptual diagram showing a schematic configuration of a medical service support system;

FIG. 2 is a flowchart showing an example of a flow of an endoscope handling service;

FIG. 3 is a block diagram showing an example of a hardware configuration of an electrical system of an endoscope part management device;

FIG. 4 is a block diagram showing an example of a function of a CPU and a storage content of a storage in an endoscope part management device main body;

FIG. 5 is a conceptual diagram showing an example of a content of an endoscopy management database;

FIG. 6 is a conceptual diagram showing an example of a content of a washer management database;

FIG. 7 is a block diagram showing an example of a hardware configuration of an electrical system of a support device server;

FIG. 8 is a block diagram showing an example of a function of a CPU and a storage content of a storage in the support device server;

FIG. 9 is a block diagram showing an example of a storage content of a management information storage device;

FIG. 10 is a flowchart showing an example of a flow of a management device control process;

FIG. 11A is a flowchart showing an example of a flow of a medical service support process according to an embodiment;

FIG. 11B is a continuation of the flowchart shown in FIG. 11A;

FIG. 11C is a continuation of the flowchart shown in FIG. 11A and FIG. 11B;

FIG. 12 is a screen view showing an example of a medical service support screen according to the embodiment; and

FIG. 13 is a screen view showing an example of the medical service support screen according to the embodiment.

### DETAILED DESCRIPTION

An example of an embodiment of a medical service support device, a medical service support method, and a program according to the present disclosed technology will be described with reference to the accompanying drawings.

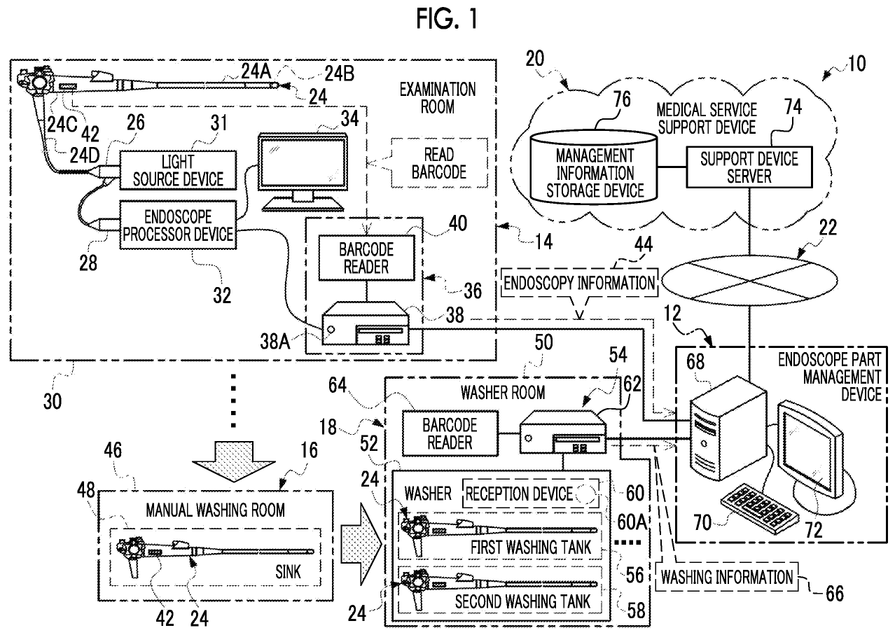

As an example, as shown in FIG. 1, a medical service support system 10 includes an endoscope part management device 12, a plurality of examination room facilities 14, a manual washing facility 16, an automatic washing facility 18, and a medical service support device 20. Here, the medical service support device 20 is an example of a “medical service support device” according to the present disclosed technology.

The medical service support system 10 is a system that supports an endoscope handling service. In the present embodiment, the endoscope handling service refers to, for example, a service performed by a person engaged in examination (hereinafter, also referred to as an “endoscopy worker”) using an endoscope 24 (hereinafter, also referred to as an “endoscopy”) in a medical installation facility (for example, a hospital). The endoscope handling service includes an endoscopy, a manual washing service, and an automatic washing service. The endoscopy is an example of an “endoscope procedure service” according to the present disclosed technology.

The medical installation facility has a plurality of endoscopes 24, and a type of endoscope 24, which corresponds to an examination item, is used in each of the plurality of examination room facilities 14. Examples of the type of endoscope 24 include an upper gastrointestinal scope, a pancreatic duct scope, a bile duct scope, a duodenal scope, a colon scope, an intracerebral scope, an otolaryngology scope, a bronchial scope, and the like.

The endoscope 24 includes an insertion part 24A, an imaging part 24B, an operation part 24C, and a universal cord 24D. The insertion part 24A is inserted into a subject.

The imaging part 24B is provided at a distal end part of the insertion part 24A. The imaging part 24B includes a complementary metal oxide semiconductor (CMOS) type imaging element. A charge-coupled device (CCD) type imaging element may be used instead of the CMOS type imaging element.

The operation part 24C is installed to be continuous with a proximal end side of the insertion part 24A, and a doctor holds the operation part 24C and performs various operations. By operating the operation part 24C, the insertion part 24A is bendable or imaging is performed by the imaging part 24B.

The universal cord 24D is installed to be continuous with the operation part 24C. The universal cord 24D encompasses a light guide (not shown), a fluid tube (not shown), and a signal cable (not shown). Connectors 26 and 28 are provided at an end part of the universal cord 24D. The connector 28 is branched from the connector 26.

A plurality of examination room facilities 14 are provided in the medical installation facility, and each of the examination room facilities 14 includes an examination room 30, a light source device 31, an endoscope processor device 32, a display 34, and an information processing apparatus 36. The light source device 31, the endoscope processor device 32, the display 34, and the information processing apparatus 36 are installed in the examination room 30.

A plurality of information processing apparatuses 36 are present and are provided in each of a plurality of medical sites where the endoscope handling service is performed. In the present embodiment, one information processing apparatus 36 is assigned to each of the examination rooms 30.

The information processing apparatus 36 is an example of an “information processing apparatus” according to the present disclosed technology and is used together with the endoscope 24 in the endoscopy. The information processing apparatus 36 operates every time an endoscopy is performed on one subject in each examination room 30. The information processing apparatus 36 is continuously operated while an endoscopy is performed on one subject. That is, a period from when the operation of the information processing apparatus 36 is started to when the operation of the information processing apparatus 36 is ended corresponds to a period during which an endoscopy is performed on one subject.

The connector 26 is connected to the light source device 31. In a case where the connector 26 is connected to the light source device 31, a light guide and a fluid tube in the universal cord 24D are inserted into the light source device 31. The light source device 31 supplies illumination light, water, and gas to the endoscope 24 via the light guide and the fluid tube. Accordingly, the illumination light is emitted toward a test observation site from an illumination window (not shown) on a distal end surface of the insertion part 24A. Further, water and gas are injected from a nozzle (not shown) on the distal end surface of the insertion part 24A toward the observation window according to the operation performed on the operation part 24C. Here, although an example of the embodiment has been described in which the light source device 31 supplies water and gas to the endoscope 24, this is merely an example, and at least one of water or gas may be supplied to the endoscope 24 from a device other than the light source device.

The connector 28 is connected to the endoscope processor device 32. In a case where the connector 28 is connected to the endoscope processor device 32, a signal cable in the universal cord 24D is electrically connected to the endoscope processor device 32. The endoscope processor device 32 controls the operation of the endoscope 24 by supplying a control signal to the endoscope 24 via a signal cable. Further, the endoscope 24 outputs an imaging signal, which is obtained by causing the imaging part 24B to image the test observation site, to the endoscope processor device 32 via the signal cable.

The endoscope processor device 32 generates a moving image as an image showing the test observation site based on the imaging signal input from the endoscope 24. A frame rate of the moving image is, for example, 30 frames per second (fps). Further, the endoscope processor device 32 generates a still image as an image showing the test observation site in response to an operation performed on the operation part 24C. The endoscope processor device 32 is connected to the display 34 and displays an image, which is generated based on the imaging signal, on the display 34.

In the present embodiment, although the light source device 31 and the endoscope processor device 32 are separate from the information processing apparatus 36, this is merely an example, and at least one of the light source device 31 or the endoscope processor device 32 may be integrated with the information processing apparatus 36.

The information processing apparatus 36 includes an information processing apparatus main body 38 and a barcode reader 40. The information processing apparatus main body 38 is configured to include a computer including a processor and a storage device. Examples of the processor include a central processing unit (CPU). Examples of the storage device include a combination of a non-volatile memory such as an electrically erasable and programmable read only memory (EEPROM), a solid state drive (SSD), and/or a hard disk drive (HDD), and a volatile memory such as a random access memory (RAM).

An examination start button 38A is provided on the information processing apparatus main body 38. The examination start button 38A is an alternate type push button. Further, the examination start button 38A is turned on at a time of starting an endoscopy for one subject and is turned off at a time of ending an endoscopy on one subject. In a case where the examination start button 38A is turned on, the information processing apparatus main body 38 starts the operation, and in a case where the examination start button 38A is turned off, the information processing apparatus main body 38 stops the operation.

The endoscope part management device 12 is connected to the information processing apparatus main body 38. An information processing apparatus ID, which is an identification (ID) capable of specifying the information processing apparatus main body 38, is assigned to the information processing apparatus main body 38, and the endoscope part management device 12 centrally manages each information processing apparatus main body 38, which is installed in each of the examination rooms 30, by using the information processing apparatus ID.

Further, the endoscope processor device 32 is connected to the information processing apparatus main body 38. That is, the endoscope 24 is connected to the information processing apparatus main body 38 via the endoscope processor device 32. Further, a barcode reader 40 is also connected to the information processing apparatus main body 38.

The information processing apparatus main body 38 is operated under the control of the endoscope part management device 12 and controls the operations of the endoscope processor device 32 and the barcode reader 40. The information processing apparatus main body 38 acquires an image, which is generated based on an imaging signal by the endoscope processor device 32, and outputs the acquired image to the endoscope part management device 12.

A barcode 42 is provided on the endoscope 24. In the example shown in FIG. 1, the barcode 42 is provided on the operation part 24C. The barcode 42 is information in which an endoscope ID that is capable of uniquely specifying the endoscope 24 is encrypted. The barcode 42 is read by the barcode reader 40 in a case where an endoscopy is started. The barcode reader 40 outputs the endoscope ID obtained by reading the barcode 42 to the information processing apparatus main body 38.

The barcode reader 40 is also capable of reading a barcode (not shown) that is uniquely assigned to the subject. The barcode, which is assigned to the subject (hereinafter, also referred to as a “subject barcode”), is information in which a subject ID that is capable of uniquely specifying the subject is encrypted, and is attached to, for example, a band (for example, a wristband) attached to the subject's wrist or the like. The subject barcode is read by the barcode reader 40 at a time at which an endoscopy is started.

The information processing apparatus main body 38 outputs various information related to the subject specified through the subject ID and endoscopy information 44 associated with various IDs to the endoscope part management device 12. Here, the various IDs refer to the endoscope IDs obtained by reading the barcode 42 with the barcode reader 40, and the subject IDs obtained by reading the subject barcode with the barcode reader 40. Here, the endoscope IDs and the subject IDs are exemplified as various IDs, but the present disclosed technology is not limited to this, and only the endoscope IDs may be used.

Further, examples of various types of information related to the subject include, for example, an image generated by the endoscope processor device 32, an examination start time point, an examination end time point, and the like. The information processing apparatus main body 38 includes a clock (for example, a real-time clock) and acquires the examination start time point and the examination end time point from the clock. For example, the examination start time point is a time point when the examination start button 38A is turned on, and the examination end time point is a time point when the examination start button 38A is turned off.

The manual washing facility 16 includes a manual washing room 46 and a sink 48. The sink 48 is installed in the manual washing room 46, and manual washing of the endoscope 24 is performed in the sink 48. Here, the manual washing refers to work in which an endoscopy worker manually and physically removes stains on the endoscope 24 by using a detergent, a brush, a sponge, or the like.

The automatic washing facility 18 includes a washer room 50, a plurality of washers 52, and a washer management device 54. The plurality of washers 52 and the washer management devices 54 are installed in the washer room 50. The washer 52 is a device that mechanically washes the endoscope 24 on which manually washing is performed. The plurality of washers 52 include a single-endoscope washer that is capable of washing only one endoscope 24 and a dual-endoscope washer that is capable of washing two endoscopes 24 together. The single-endoscope washer is also conventionally referred to as a single washer, and the dual-endoscope washer is also conventionally referred to as a dual washer.

The single-endoscope washer is provided with only one washing tank into which one endoscope 24 is inserted, whereas the dual-endoscope washer is provided with a first washing tank 56 and a second washing tank 58. One endoscope 24 is inserted into each of the first washing tank 56 and the second washing tank 58. Hereinafter, in a case where it is not necessary to distinguish among the washing tank of the single-endoscope washer, the first washing tank 56, and the second washing tank 58, a term “washing tank” will be used without reference numerals.

The washer 52 includes a reception device 60. The reception device 60 includes a plurality of buttons and a dial. The plurality of buttons include a washing start button 60A. The washing start button 60A is a button that receives an instruction for causing the washer 52 to start washing the endoscope 24 in the washing tank. In a case where the washing start button 60A is turned on, the washer 52 starts the operation. In a case where the washer 52 is operated, the washer 52 sequentially performs washing, rinsing, dehydration, and the like with a detergent for the endoscope 24 in the washing tank according to a default program.

The washing time by the washer 52, that is, the operation time of the washer 52, is designated by operating the dial included in the reception device 60. After a lapse of time designated by operating the dial, the washer 52 stops the operation.

The washer management device 54 includes a washer management device main body 62 and a barcode reader 64. The washer management device main body 62 is configured to include a computer including a processor and a storage device. Examples of the processor include a CPU. Examples of the storage device include a combination of a non-volatile memory such as an EEPROM, an SSD, and/or an HDD, and a volatile memory such as a RAM.

The plurality of washers 52 are connected to the washer management device main body 62, and the washer management device main body 62 centrally manages the plurality of washers 52. For example, the washer management device main body 62 includes a clock and acquires a washing start time point and a washing end time point of each of the plurality of washers 52 from the clock. The washing start time point is a time point when the washing start button 60A of the washer 52 is turned on, and the washing end time point is a time point when the operation of the washer 52 is stopped (for example, a time point when washing of the endoscope 24 in the washing tank is ended).

Here, although an example of the embodiment has been described in which the plurality of washers 52 are centrally managed by the washer management device 54, this is merely an example, and each of a plurality of washer management devices 54 may individually manage the plurality of washers 52, and one washer management device 54 may centrally manage the plurality of washers 52.

Further, here, although a time point when the washing start button 60A is turned on is exemplified as the washing start time point, this is merely an example, and, for example, the washing start time point may be determined according to an instruction given to a device other than the washer management device 54 such as the endoscope part management device 12. Further, here, although a time point when the operation of the washer 52 is stopped is exemplified as the washing end time point, this is merely an example, and the washing end time point may be a time point when a timer for managing the washing time is ended, or a time point when the washing start button 60A is turned off.

A washer ID, which is an ID that is capable of specifying the washer 52, is assigned to each of the plurality of washers 52, and the washer management device main body 62 centrally manages the plurality of washers 52 by using washer IDs.

The barcode reader 64 is connected to the washer management device main body 62. The endoscope part management device 12 is connected to the washer management device main body 62, and the washer management device main body 62 is operated under the control of the endoscope part management device 12 and controls the operation of the plurality of washers 52 and the barcode reader 64.

The barcode 42 of the endoscope 24, on which manual washing is performed, is read by the barcode reader 64. The barcode reader 64 outputs the endoscope ID obtained by reading the barcode 42 to the washer management device main body 62.

The washer management device main body 62 outputs various types of information related to the washer 52 and washing information 66 associated with the endoscope ID corresponding to the endoscope 24 washed by the washer 52 to the endoscope part management device 12. Here, examples of various types of information related to the washer 52 include a washer ID, washing start time point, and washing end time point.

Each of the endoscope 24, the information processing apparatus 36, and the washer 52 is an example of a “medical device used in the endoscope handling service that includes an endoscope procedure service” according to the present disclosed technology. Further, in the following, for convenience of explanation, in a case where it is not necessary to distinguish among the endoscope 24, the information processing apparatus 36, and the washer 52, a term “medical device” will also be used without reference numerals.

The endoscope part management device 12 is a device that manages a part (endoscope part) that is responsible for the endoscope handling service in a medical installation facility. The endoscope part management device 12 includes an endoscope part management device main body 68, a reception device 70, and a display 72. The display 72 is an example of a “presentation device” according to the present disclosed technology.

As described in detail later, the endoscope part management device main body 68 is a device configured to include a computer. The reception device 70 is connected to the endoscope part management device main body 68. The reception device 70 includes a keyboard, a mouse, a touch panel, and/or the like, and receives an instruction from a user or the like of the endoscope part management device 12. The display 72 is connected to the endoscope part management device main body 68. The display 72 displays various types of information under the control of the endoscope part management device main body 68. Examples of the display 72 include an electro-luminescence (EL) display, a liquid crystal display, and the like.

The endoscope part management device main body 68 acquires the endoscopy information 44 output from the information processing apparatus main body 38 and the washing information 66 output from the washer management device main body 62, and executes a process by using the acquired endoscopy information 44 and washing information 66. In the following, for convenience of explanation, in a case where it is not necessary to distinguish between the endoscopy information 44 and the washing information 66, a term “management target information” will be used without reference numerals.

In the present embodiment, the endoscope part management device 12 is connected to the medical service support device 20 via a network 22. The network 22 is, for example, the Internet. Note that the network 22 is not limited to the Internet and may be a wide area network (WAN) and/or a local area network (LAN) such as an intranet. Further, the endoscope part management device 12 and the medical service support device 20 may be integrally formed, and the medical service support device 20 may have at least some of the functions of the endoscope part management device 12.

The endoscope part management device main body 68 exchanges information with the medical service support device 20 via the network 22. The medical service support device 20 is realized by cloud computing. Here, although cloud computing is exemplified, this is merely an example, and, for example, the medical service support device 20 may be implemented by a mainframe or implemented by network computing such as fog computing, edge computing, or grid computing.

The medical service support device 20 includes a support device server 74 and a management information storage device 76. The management information storage device 76 is connected to the support device server 74. The support device server 74 is connected to the endoscope part management device main body 68 via the network 22, performs a process in response to a request from the endoscope part management device main body 68, and provides a processing result to the endoscope part management device main body 68.

The endoscope part management device main body 68 anonymizes the endoscopy information 44 and transmits the anonymized endoscopy information 44 and washing information 66 to the support device server 74. The anonymization of the endoscopy information 44 means the anonymization of the subject specified based on the subject ID included in the endoscopy information 44.

The support device server 74 receives the endoscopy information 44 and the washing information 66, and stores the received endoscopy information 44 and the washing information 66 in the management information storage device 76. The support device server 74 selectively acquires the endoscopy information 44 and the washing information 66 from the management information storage device 76 as necessary, and executes a process by using the acquired information.

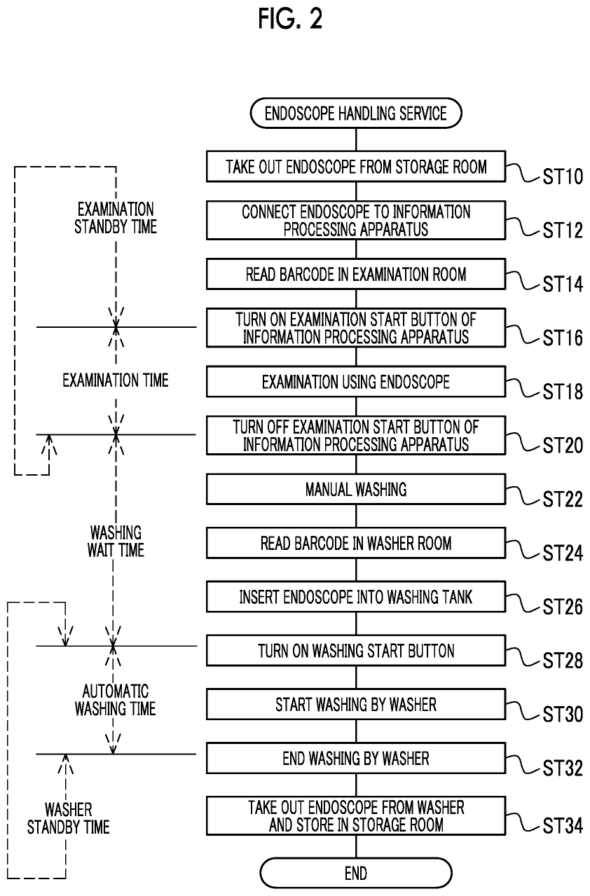

FIG. 2 shows an example of a flow of an endoscope handling service performed by the endoscopy worker. In the following, for convenience of explanation, the description is based on the premise that the usable endoscope 24 (for example, the endoscope 24 washed by the washer 52) is stored in a storage room (not shown) in a medical installation facility by the endoscopy worker.

In the endoscope handling service shown in FIG. 2, first, in step ST10, the endoscope 24 is taken out from the storage room by the endoscopy worker, and the endoscope 24 is transported into one examination room 30 among the plurality of examination rooms 30.

In the next step ST12, the endoscope 24 is connected to the information processing apparatus 36 in the examination room 30 via the light source device 31 and the endoscope processor device 32.

In the next step ST14, the barcode 42 of the endoscope 24 is read by the barcode reader 40 in the examination room 30. Accordingly, the endoscope ID of the endoscope 24 is acquired by the information processing apparatus 36.

In the next step ST16, the examination start button 38A of the information processing apparatus 36 is turned on. Further, an examination start button (not shown) is also provided in the endoscope processor device 32, and the examination start button of the endoscope processor device 32 is turned on. Accordingly, an endoscopy is started. Here, although an example of the embodiment has been described in which a process of step ST14 is performed prior to a process of step ST16, this is merely an example, and the process of step ST16 may be performed prior to the process of step ST14. That is, the order of step ST14 and step ST16 may be reversed.

In the following, for convenience of explanation, although the description is based on the premise that an endoscopy is started on a condition that the examination start button 38A is turned on, the present disclosed technology is not limited to this. For example, the endoscope processor device 32 may also include an examination start button similar to the examination start button 38A of the information processing apparatus 36, and the endoscopy may be started on a condition that the examination start button 38A of the information processing apparatus 36 is turned on and the examination start button of the endoscope processor device 32 is also turned on. In this case, the endoscope processor device 32 and the information processing apparatus 36 are examples of the “information processing apparatuses” according to the present disclosed technology.

In the next step ST18, an examination using the endoscope 24 (such as imaging of a test observation site by the imaging part 24B of the endoscope 24) is performed by a doctor or the like. Accordingly, the information processing apparatus 36 generates an image based on an imaging signal obtained by imaging by the imaging part 24B.

In the next step ST20, the examination start button 38A of the information processing apparatus 36 is turned off. Further, the examination start button of the endoscope processor device 32 is also turned off. Accordingly, the endoscopy is ended. The time during which the information processing apparatus 36 is being operated corresponds to time from when the examination start button 38A is turned on in step ST16 to when the examination start button 38A is turned off in step ST20, and is time (hereinafter, also referred to as “examination time”) during which the endoscopy is being performed.

In the following, for convenience of explanation, although the description is based on the premise that the endoscopy is ended on a condition that the examination start button 38A is turned off, the present disclosed technology is not limited to this. For example, the endoscopy may end on a condition that the examination start button 38A of the information processing apparatus 36 is turned off and the examination start button of the endoscope processor device 32 is also turned off.

In the next step ST22, manual washing of the endoscope 24 used in step ST18 is performed. In the next step ST24, the barcode 42 of the endoscope 24 manually washed in the washer room 50 is read by the barcode reader 64. Accordingly, the endoscope ID of the endoscope 24 is acquired by the washer management device 54.

In the next step ST26, the endoscope 24 is inserted into the washing tank of the washer 52. In the next step ST28, the washing start button 60A is turned on.

Note that time from when the endoscopy is ended to when washing of the endoscope 24 by the washer 52 is started, that is, time from when the examination start button 38A is turned off in step ST20 to when the washing start button 60A is turned on in step ST28, is washing wait time (hereinafter, also simply referred to as “washing wait time”) to when washing of the endoscope 24 by the washer 52 is started. The washing wait time also includes time during which manual washing is performed on the endoscope 24 (hereinafter, also referred to as “manual washing time”). In the present embodiment, in the following, for convenience of explanation, the washing wait time will be described as a manual washing time.

In the next step ST30, washing of the endoscope 24, which is inserted into the washing tank in step ST26, is started by the washer 52. In the next step ST32, after a lapse of designated time after the washing start button 60A is turned on in step ST28, the washing of the endoscope 24, which is inserted into the washing tank in step ST26, by the washer 52 is ended, and one endoscope handling service is ended.

The time from when the washing start button 60A is turned on in step ST28 to when the washing of the endoscope 24 by the washer 52 is ended in step ST32 is time corresponding to time during which washing of the endoscope 24 is being performed by the washer 52 (hereinafter, also referred to as “automatic washing time”).

In the next step ST34, the endoscope 24 is taken out from the washer 52, and the taken-out endoscope 24 is stored in the storage room.

In a case where N is a natural number, in a specific examination room 30, time required from immediately before a time when an N-th endoscopy is ended (immediately before a time when the examination start button 38A is turned off in step ST20 included in the N-th endoscope handling service) to immediately before a time when an (N+1)-th endoscopy is started (immediately before a time when the examination start button 38A is turned on in step ST16 included in the (N+1)-th endoscope handling service) is time corresponding to time during which the endoscopy is on standby in a specific examination room 30 (hereinafter, also referred to as “examination standby time”).

Further, in the washer room 50, time that is required from when the N-th washing by the washer 52 is ended to when the (N+1)-th washing by the washer 52 is started (restarted), is time corresponding to time during which the washer 52 is on standby for washing of the endoscope 24 (hereinafter, also referred to as “washer standby time”). Here, a target to be washed by the washer 52 is an unspecified endoscope 24. However, this is merely an example, and the specific endoscope 24 may be washed by the specific washer 52.

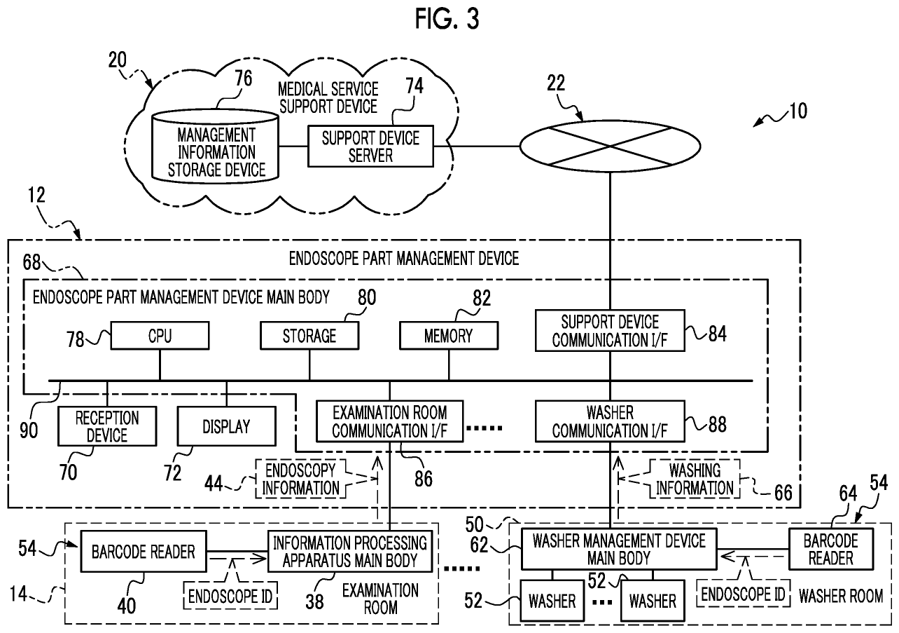

As an example, as shown in FIG. 3, the endoscope part management device main body 68 includes a CPU 78, a storage 80, a memory 82, a support device communication (interface) I/F 84, a plurality of examination room communication I/Fs 86, and a washer communication I/F 88. The CPU 78, the storage 80, the memory 82, the support device communication I/F 84, the plurality of examination room communication I/Fs 86, and the washer communication I/F 88 are connected to a bus 90.

The CPU 78 controls the entire endoscope part management device main body 68. The storage 80 is a non-volatile storage device that stores various programs, various parameters, and the like. Examples of the storage 80 include an EEPROM, an SSD, and/or an HDD. The memory 82 is a memory in which information is temporarily stored and is used as a work memory by the CPU 78. Examples of the memory 82 include a RAM.

The support device communication I/F 84 is connected to the network 22 and controls the exchange of information between the CPU 78 and the support device server 74. For example, the support device communication I/F 84 transmits information in response to a request from the CPU 78 to the support device server 74 via the network 22, receives the information transmitted from the support device server 74, and outputs the received information to the CPU 78.

The examination room communication I/F 86 is provided for each examination room 30 and is connected to the information processing apparatus main body 38 in the examination room 30. The examination room communication I/F 86 controls the exchange of information between the CPU 78 and the information processing apparatus main body 38. For example, the examination room communication I/F 86 transmits information in response to a request from the CPU 78 to the information processing apparatus main body 38 designated by the CPU 78 among the plurality of information processing apparatus main bodies 38. Further, the examination room communication I/F 86 acquires the endoscopy information 44 from the information processing apparatus main body 38 and outputs the acquired endoscopy information 44 to the CPU 78.

The washer communication I/F 88 is connected to a washer management device main body 62 and controls the exchange of information between the CPU 78 and the washer management device main body 62. For example, the washer communication I/F 88 transmits information in response to a request from the CPU 78 to the washer management device main body 62. Further, the washer communication I/F 88 acquires the washing information 66 from the washer management device main body 62 and outputs the acquired washing information 66 to the CPU 78.

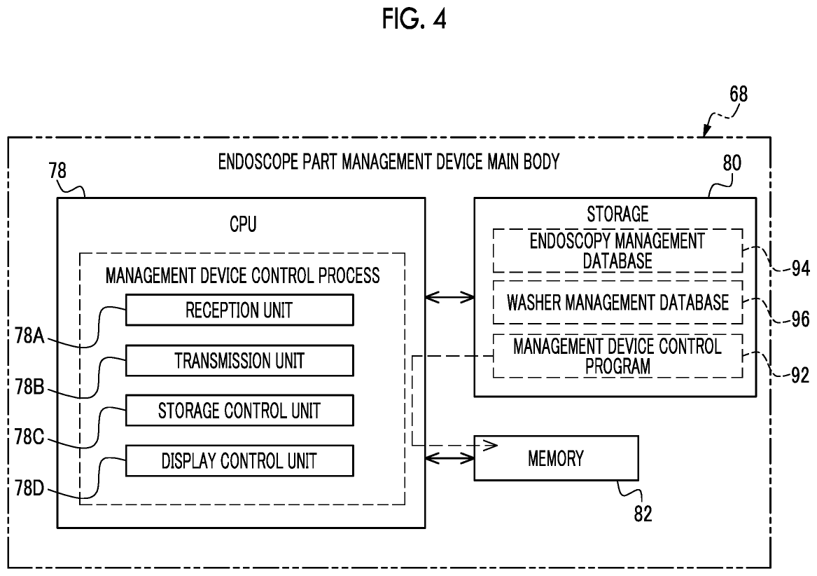

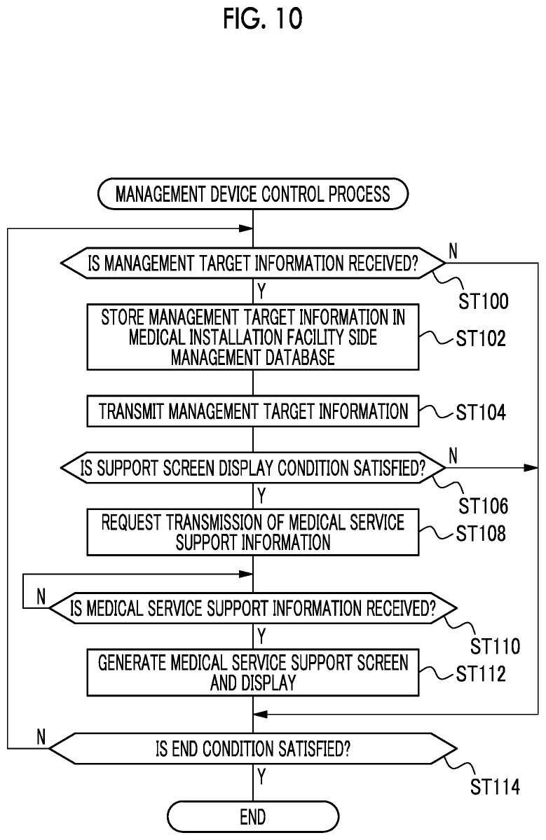

As an example, as shown in FIG. 4, in the endoscope part management device main body 68, the storage 80 stores a management device control program 92. The CPU 78 reads the management device control program 92 from the storage 80 and performs a management device control process by executing the read management device control program 92 on the memory 82 (see FIG. 10). The management device control process is realized by the CPU 78 operating as a reception unit 78A, a transmission unit 78B, a storage control unit 78C, and a display control unit 78D in accordance with the management device control program 92. Specific processing contents of the reception unit 78A, the transmission unit 78B, the storage control unit 78C, and the display control unit 78D will be described later with reference to FIG. 10.

An endoscopy management database 94 and a washer management database 96 are constructed in the storage 80. The storage control unit 78C acquires the endoscopy information 44 (see FIG. 1 and FIG. 3) via the examination room communication I/F 86 (see FIG. 3) and stores the acquired endoscopy information 44 in the endoscopy management database 94. Further, the storage control unit 78C acquires the washing information 66 (see FIG. 1 and FIG. 3) via the washer communication I/F 88 (see FIG. 3) and stores the acquired washing information 66 in the washer management database 96. In the following, for convenience of explanation, in a case where it is not necessary to distinguish between the endoscopy management database 94 and the washer management database 96, a term “medical installation facility side management database” will also be used.

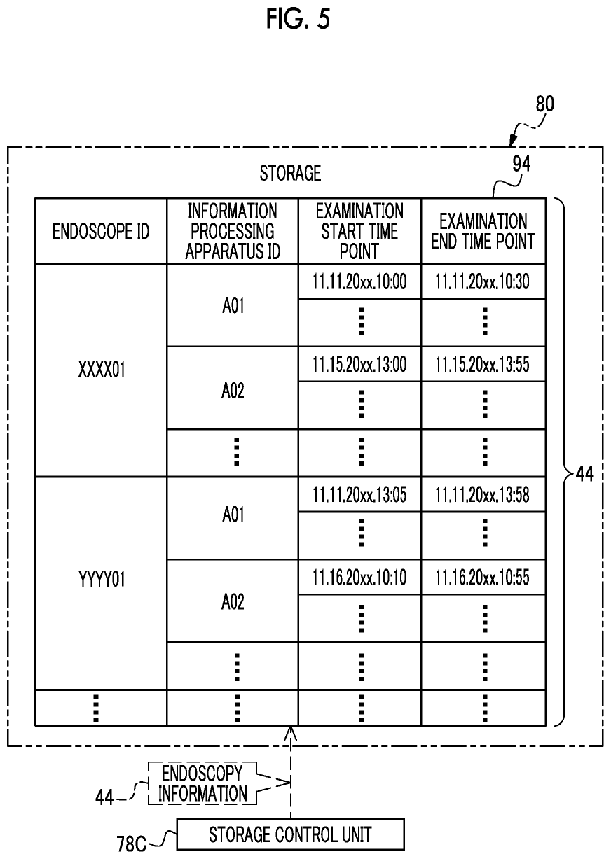

As an example, as shown in FIG. 5, in the endoscopy management database 94, the endoscopy information 44 acquired by the storage control unit 78C from each information processing apparatus main body 38 installed in each of the plurality of examination rooms 30 is stored. In the endoscopy management database 94, the examination start time point and the examination end time point are associated for each endoscope ID and for each information processing apparatus ID. Further, the subject ID is associated with the examination start time point or the examination end time point (not shown).

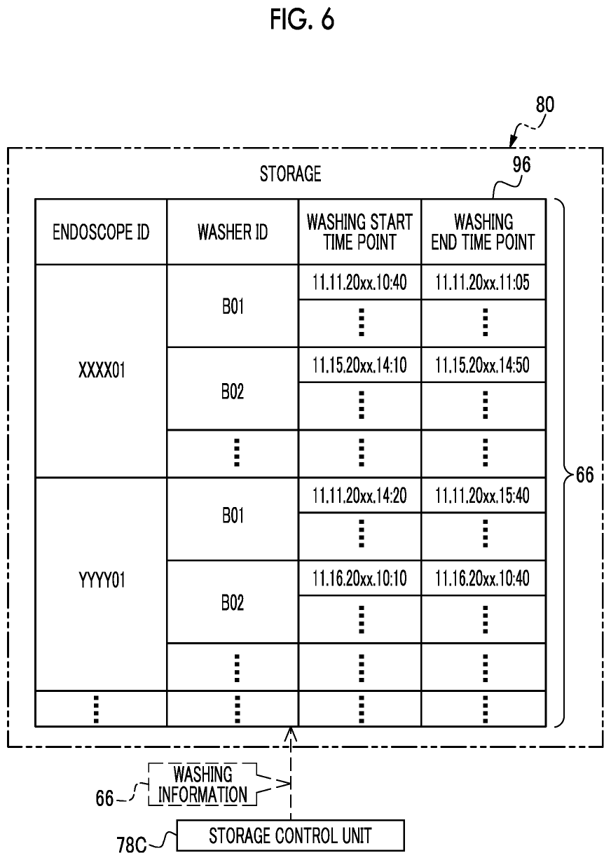

As an example, as shown in FIG. 6, in the washer management database 96, the washing information 66 acquired by the storage control unit 78C from the washer management device main body 62 is stored. In the washer management database 96, the washing start time point and the washing end time point are associated for each endoscope ID and for each washer ID.

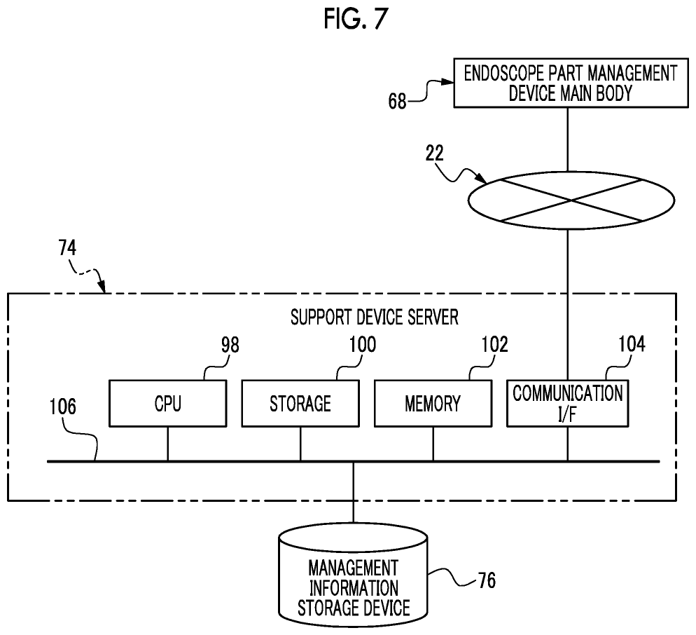

As an example, as shown in FIG. 7, the support device server 74 includes a CPU 98, a storage 100, a memory 102, and a communication I/F 104. The CPU 98 is an example of a “processor” according to the present disclosed technology, and the memory 102 is an example of a “memory” according to the present disclosed technology.

The CPU 98, the storage 100, the memory 102, and the communication I/F 104 are connected to a bus 106. Further, the management information storage device 76 is connected to the bus 106. Examples of the management information storage device 76 include an EEPROM, an SSD, and/or an HDD. In the management information storage device 76, anonymized management target information is stored.

The CPU 98 controls the entire support device server 74. The storage 100 is a non-volatile storage device that stores various programs, various parameters, and the like. Examples of the storage 100 include an EEPROM, an SSD, and/or an HDD. The memory 102 is a memory in which information is temporarily stored and is used as a work memory by the CPU 98. Examples of the memory 102 include a RAM.

The communication I/F 104 is connected to the endoscope part management device main body 68 via the network 22 and controls the exchange of information between the CPU 98 and the endoscope part management device main body 68. For example, the communication I/F 104 receives the information transmitted from the endoscope part management device main body 68 and outputs the received information to the CPU 78. Further, the communication I/F 104 transmits information in response to a request from the CPU 98 to the endoscope part management device main body 68 via the network 22.

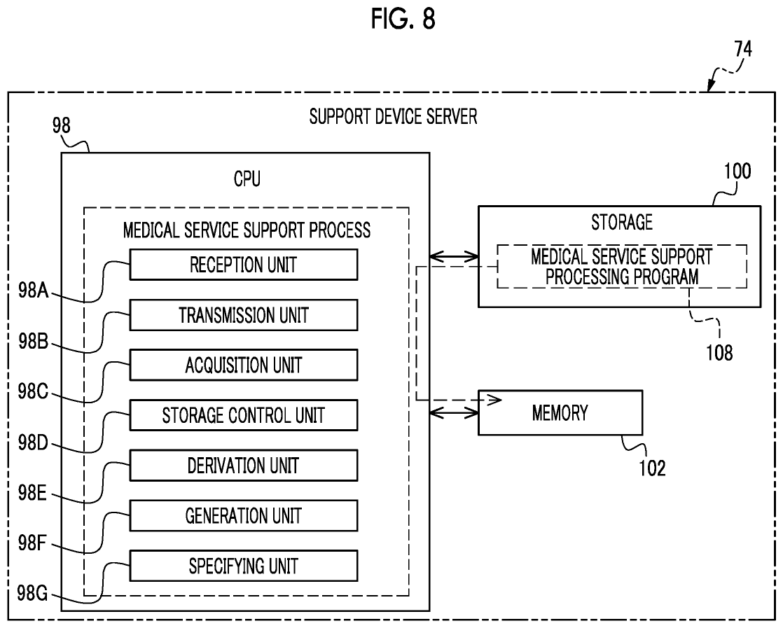

As an example, as shown in FIG. 8, in the support device server 74, the storage 100 stores a medical service support processing program 108. The support device server 74 is an example of a “computer” according to the present disclosed technology, and the medical service support processing program 108 is an example of a “program” according to the present disclosed technology.

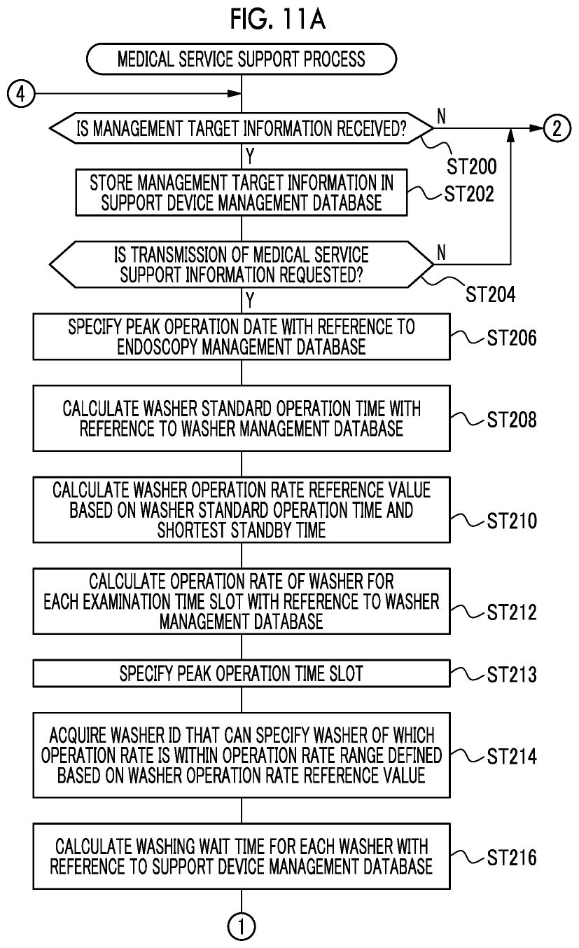

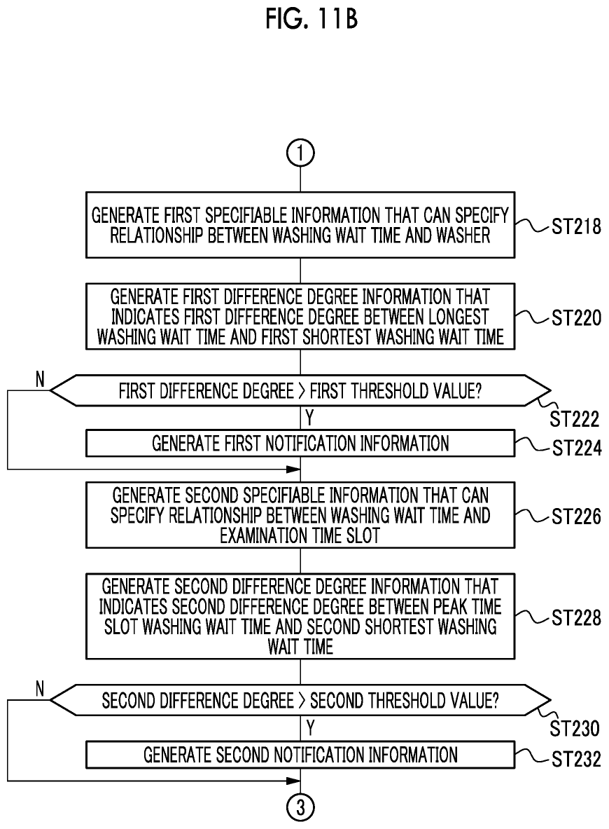

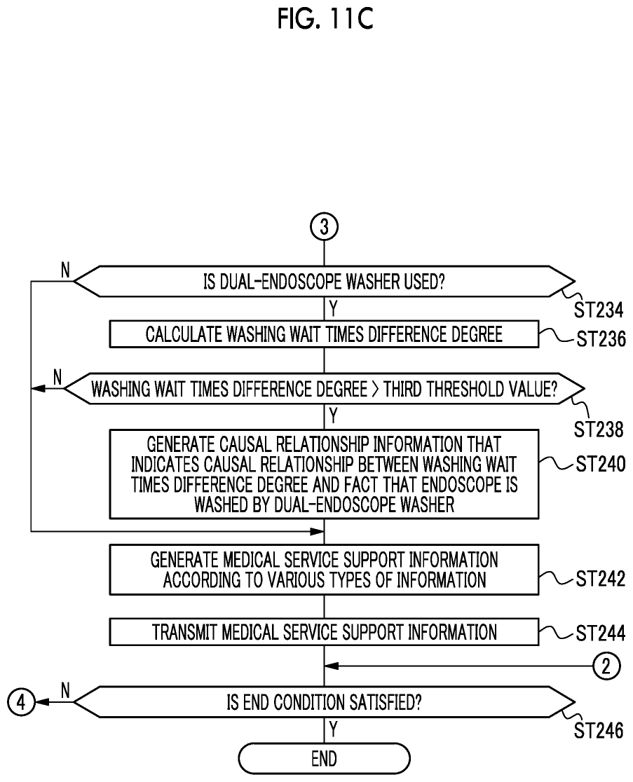

The CPU 98 reads the medical service support processing program 108 from the storage 100 and performs a medical service support process by executing the read medical service support processing program 108 on the memory 102 (see FIG. 11A to FIG. 11C). The medical service support process is realized by the CPU 98 operating as a reception unit 98A, a transmission unit 98B, an acquisition unit 98C, a storage control unit 98D, a derivation unit 98E, a generation unit 98F, a specifying unit 98G in accordance with the medical service support processing program 108. Specific processing contents of the reception unit 98A, the transmission unit 98B, the acquisition unit 98C, the storage control unit 98D, the derivation unit 98E, the generation unit 98F, the specifying unit 98G will be described later with reference to FIG. 11A to FIG. 11C.

By performing the medical service support process, the CPU 98 acquires the washing wait time for each default unit capable of classifying the washing wait time and outputs the medical service support information in which the washing wait time and the default unit are associated with each other. The acquisition of the washing wait time by the CPU 98 is performed for each of the plurality of washers 52 installed in the washer room 50.

A first example of the default unit is a medical device unit. The medical device unit indicates, for example, a unit classified by related information that can identify a predetermined medical device from a plurality of medical devices. Examples of the related information that can identify the medical device include related information that is related to the washer 52. In the present embodiment, the related information that is related to the washer 52 refers to the washer ID. The washer ID is, for example, a serial number of the washer 52. The washer ID is merely an example, and the related information that is related to the washer 52 may be information including information capable of specifying that the washer 52 is a single-endoscope washer, information capable of specifying that the washer 52 is a dual-endoscope washer, information capable of specifying which sales series the washer 52 belongs to, and/or information capable of specifying the manufacturer of the washer 52, instead of the washer ID or together with the washer ID. In other words, the related information that can identify the predetermined medical device may be the related information that can identify medical devices having the same function, medical devices made by the same manufacturer, or medical devices with the same sales series.

A second example of the default unit is a time slot unit. The time slot unit indicates a unit classified by a predetermined time. The time slot may be limited to a time slot during which the medical device operates or is likely to operate and may be the examination time slot, for example. The examination time slot is an example of the “time slot” of the present disclosed technology. In the present embodiment, the examination time slot refers to a time slot during which the endoscopy is performed. An example of the examination time slot includes a time slot from 9:00 to 16:00 in units of one hour. The time slot from 9:00 to 16:00 in units of one hour is merely an example, and may be, for example, a time slot of several minutes to several hours within the time of outpatient treatment at a medical installation facility. In other words, the predetermined time may be a time slot in any unit from several minutes to several hours, and the time slot in which medical time is in operation or a time slot in which the medical time may be in operation may be at least a part of the examination time slot or the outpatient treatment time slot at the medical installation facility.

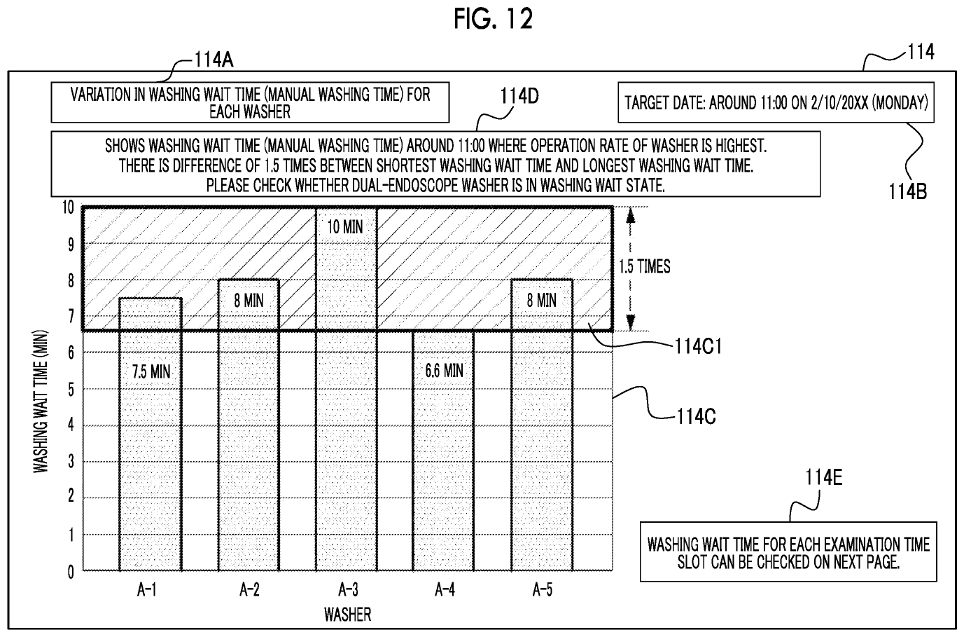

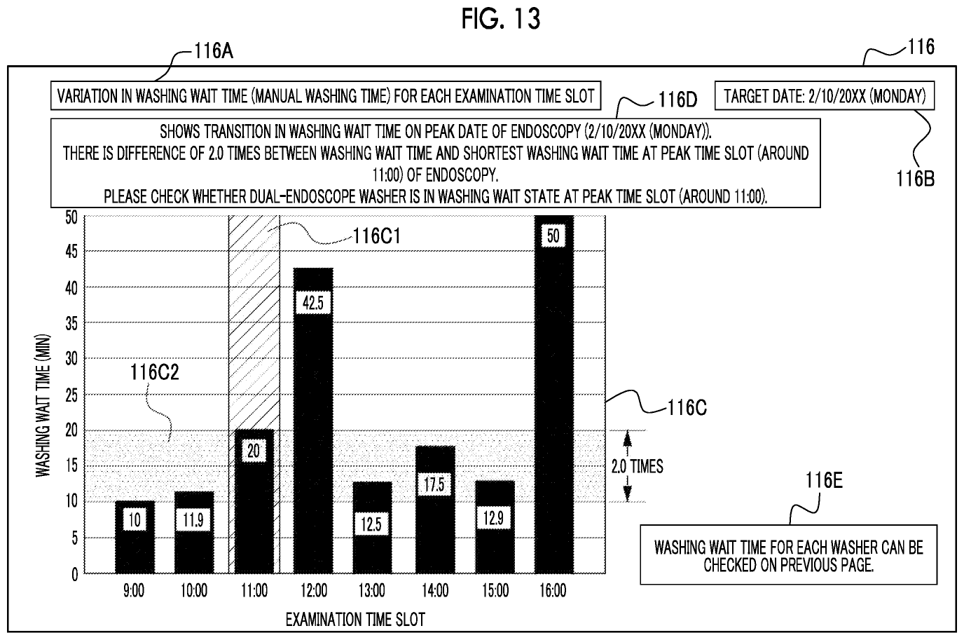

As described in detail later, the medical service support information is information that includes information capable of specifying a relationship between the washing wait time and the default unit. The medical service support information includes screen information showing a medical service support screen 114 (see FIG. 12) and a medical service support screen 116 (see FIG. 13) as information capable of specifying the relationship between the washing wait time and the default unit.

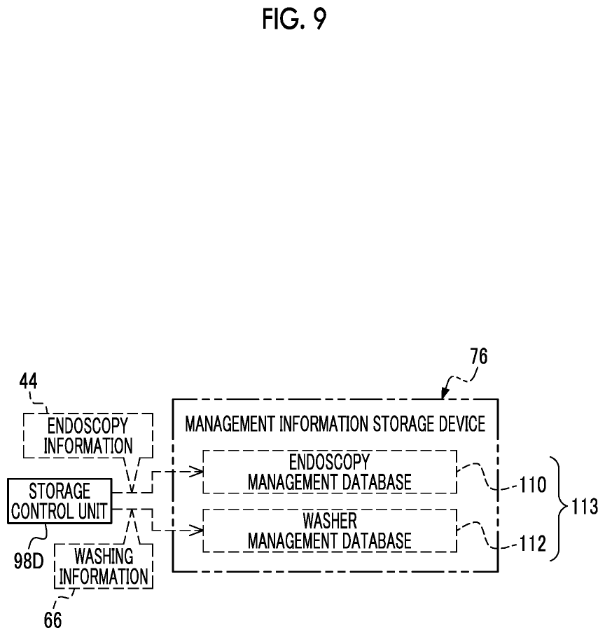

As an example, as shown in FIG. 9, a support device management database 113 is constructed in the management information storage device 76. The support device management database 113 includes an endoscopy management database 110 and a washer management database 112. The anonymized endoscopy information 44 is stored in the endoscopy management database 110, and the washing information 66 is stored in the washer management database 112.

The storage control unit 98D acquires the anonymized endoscopy information 44 from the endoscope part management device main body 68 via the communication I/F 104 (see FIG. 7) and stores the acquired endoscopy information 44 in the endoscopy management database 110. The storage control unit 98D acquires the washing information 66 (see FIG. 6) from the endoscope part management device main body 68 via the communication I/F 104 (see FIG. 7) and stores the acquired washing information 66 in the washer management database 112.

Next, the operation of the medical service support system will be described.

FIG. 10 shows an example of a flow of a management device control process performed by the CPU 78 of the endoscope part management device 12. In the present embodiment, the information processing apparatus 36 will be described below as an example of the medical device.

In the management device control process shown in FIG. 10, first, in step ST100, the reception unit 78A determines whether or not the management target information is received through the examination room communication I/F 86 (see FIG. 3) or the washer communication I/F 88 (see FIG. 3). In step ST100, in a case in which the management target information is not received through the examination room communication I/F 86 or the washer communication I/F 88, a negative determination is made, and the management device control process proceeds to step ST114. In step ST100, in a case in which the management target information is received through the examination room communication I/F 86 or the washer communication I/F 88, a positive determination is made, and the management device control process proceeds to step ST102.

In step ST102, the storage control unit 78C stores the management target information, which is received through the examination room communication I/F 86 or the washer communication I/F 88 in step ST100, in the medical installation facility side management database. That is, the endoscopy information 44 is stored in the endoscopy management database 94 (see FIGS. 4 and 5), and the washing information 66 is stored in the washer management database 96 (see FIGS. 4 and 6). After the process in step ST102 is executed, the management device control process proceeds to step ST104.

In step ST104, the transmission unit 78B transmits the management target information, which is received in step ST100, to the support device server 74 via the support device communication I/F 84 (see FIG. 3). After the process in step ST104 is executed, the management device control process proceeds to step ST106.

In step ST106, the transmission unit 78B determines whether or not a support screen display condition, which is a condition for displaying the medical service support screen 114 (see FIG. 12) and the medical service support screen 116 (see FIG. 13) on the display 72 (see FIG. 1 and FIG. 3), is satisfied. A first example of the support screen display condition includes a condition that the reception device 70 (see FIGS. 1 and 3) receives an instruction for displaying the medical service support screens 114 and 116 on the display 72. Further, a second example of the support screen display condition includes a condition that a time point (for example, 22:00), which is designated in advance, has arrived. Further, the second example of the support screen display condition includes a condition that an amount of information of the management target information stored in the medical installation facility side management database reaches a default amount of information.

In step ST106, in a case where the support screen display condition is not satisfied, a negative determination is made, and the management device control process proceeds to step ST114. In step ST106, in a case where the support screen display condition is satisfied, a positive determination is made, and the management device control process proceeds to step ST108.

In step ST108, the transmission unit 78B requests transmission of the medical service support information from the support device server 74 via the support device communication I/F 84 (see FIG. 3). After the process in step ST108 is executed, the management device control process proceeds to step ST110.

In a case where the process of step ST108 is executed, the processes of step ST206 to step ST216, which are included in the medical service support process shown in FIG. 11, are executed by the support device server 74, and the medical service support information is transmitted from the support device server 74 to the endoscope part management device 12 via the network 22 by executing step ST216.

In step ST110, the reception unit 78A determines whether or not the medical service support information, which is transmitted from the support device server 74, is received through the support device communication I/F 84 (see FIG. 3). In step ST110, in a case where the medical service support information is not received through the support device communication I/F 84, a negative determination is made, and the determination in step ST110 is performed again. In step ST110, in a case in which the medical service support information is received through the support device communication I/F 84, a positive determination is made, and the management device control process proceeds to step ST112.

In step ST112, the display control unit 78D generates the medical service support screen 114 (see FIG. 12) and the medical service support screen 116 (see FIG. 13) by using the medical service support information received through the support device communication I/F 84 in step ST110 and displays the medical service support screen 114 (see FIG. 12) and the medical service support screen 116 (see FIG. 13) on the display 72 (see FIG. 1 and FIG. 3). After the process in step ST112 is executed, the management device control process proceeds to step ST114.

In step ST114, the display control unit 78D determines whether or not a condition for ending the management device control process (hereinafter, referred to as a “management device control process end condition”) is satisfied. Examples of the management device control process end condition include a condition that an instruction for ending the management device control process is received by the reception device 70 (see FIG. 1 and FIG. 3).

In step ST114, in a case where the management device control process end condition is not satisfied, a negative determination is made, and the management device control process proceeds to step ST100. In step ST114, in a case where the management device control process end condition is satisfied, the management device control process is ended.

FIG. 11A to FIG. 11C show an example of a flow of the medical service support process performed by the CPU 98 of the support device server 74.

In the medical service support process shown in FIG. 11A, first, in step ST200, the reception unit 98A determines whether or not the management target information, which is transmitted by executing the process of step ST100 included in the management device control process shown in FIG. 10, is received through the communication I/F 104 (see FIG. 7). In step ST200, in a case where the management target information is not received through the communication I/F 104, a negative determination is made, and the medical service support process proceeds to step ST246. In step ST200, in a case where the management target information is received through the communication I/F 104, a positive determination is made, and the medical service support process proceeds to step ST202.

In step ST202, the storage control unit 98D stores the management target information, which is received through the communication I/F 104 in step ST200, in the support device management database 113. That is, the endoscopy information 44 is stored in the endoscopy management database 110 (see FIG. 9), and the washing information 66 is stored in the washer management database 112 (see FIG. 9). After the process in step ST202 is executed, the medical service support process proceeds to step ST204.

In step ST204, the reception unit 98A determines whether or not transmission of the medical service support information is requested from the endoscope part management device 12 by executing the process of step ST108 included in the management device control process shown in FIG. 10. In step ST204, in a case where the transmission of the medical service support information is not requested from the endoscope part management device 12, a negative determination is made, and the medical service support process proceeds to step ST246. In step ST204, in a case where the transmission of the medical service support information is requested from the endoscope part management device 12, a positive determination is made, and the medical service support process proceeds to step ST206.

In step ST206, the specifying unit 98G specifies a date on which an operation status of the medical device peaks (hereinafter, also referred to as a “peak operation date”) with reference to the endoscopy management database 110 (see FIG. 9). After the process in step ST206 is executed, the medical service support process proceeds to step ST208. In the present embodiment, the peak operation date for the medical device refers to a date on which the operation statuses of all the information processing apparatuses 36 (hereinafter also referred to as “all information processing apparatuses 36”) installed in all the examination rooms 30 peak. The peak operation date is an example of “a time period when the operation degree of the medical device is equal to or higher than the reference degree” according to the present disclosed technology.

Here, a date on which an endoscopy is performed the highest number of times is regarded as the peak operation date. The date on which an endoscopy is performed the highest number of times is an example of “a time period when a value based on the number of endoscope procedure services is equal to or greater than a reference value” according to the present disclosed technology.

The date on which an endoscopy is performed can be specified by using the examination start time point or the examination end time point of the endoscopy information 44 stored in the endoscopy management database 110. Therefore, in the present step ST206, the date on which an endoscopy is performed the highest number of times is specified by the specifying unit 98G by using the examination start time point or the examination end time point of the endoscopy information 44 included in the endoscopy management database 110.

The peak operation date may be specified for all periods that can be specified based on the endoscopy information 44 stored in the endoscopy management database 110 or may be a period (for example, a designated number of years, a designated number of months, or a designated number of days) designated based on the endoscopy information 44 stored in the endoscopy management database 110. The designation of the period is performed, for example, by a user or the like via the reception device 70 (see FIG. 1 and FIG. 3) of the endoscope part management device 12.

In the step ST208, the derivation unit 98E calculates washer standard operation time for the peak operation date specified in step ST206 with reference to the washer management database 112. After the process in step ST208 is executed, the medical service support process proceeds to step ST210.

In step ST208, the washer standard operation time is a statistical value of time based on the operation time of the washer 52 on the peak operation date, that is, the operation time of all washers 52 (washer operation time for all the washers 52) on the peak operation date. Here, the washer operation time refers to the operation time of the washer 52. The washer operation time is the automatic washing time shown in FIG. 2, that is, time from when the washing start button 60A is turned on in step ST28 shown in FIG. 2 to when washing of the endoscope 24 by the washer 52 is ended in step ST32 shown in FIG. 2.

For example, the washer standard operation time is an average value of operation time of all the washers 52 on the peak operation date. Here, although the average value of the operation time of all the washers 52 is exemplified as the washer standard operation time, this is merely an example, and it may be a statistical value such as a median value, the most frequent value, the maximum value, the minimum value, or a percentile of the operation time of all the washers 52. Further, the washer standard operation time may be calculated within a time range (for example, within a range of substantially 10 minutes to 50 minutes) predetermined with an upper limit value and a lower limit value.

In step ST210, the derivation unit 98E calculates a washer operation rate reference value based on the washer standard operation time calculated in step ST208 and on the shortest standby time. After the process in step ST210 is executed, the medical service support process proceeds to step ST212. In the present step ST210, the shortest standby time refers to the shortest standby time from when washing of the endoscope 24 by the washer 52 is ended to when washing of the endoscope 24 by the washer 52 is started. The endoscope 24 that is washed by the washer 52 is an unspecified endoscope 24. However, this is merely an example, and the specific endoscope 24 may be washed by the specific washer 52.

Here, the shortest standby time refers to, for example, the shortest time of the standby time from the end of washing performed by each of all the washers 52 to the start of washing. Here, as an example of the shortest standby time, predetermined time (for example, pre-designated time between 1 minute and 3 minutes) is applied as washer washing time shown in FIG. 2, that is, the shortest time from when washing of the endoscope 24 by the washer 52 is ended (for example, from when the process of step ST32 shown in FIG. 2 is ended) to when washing of the endoscope 24 by the washer 52 is started (for example, to when the process of step ST28 shown in FIG. 2 is started again). The shortest standby time may be a fixed value or a variable value that is changed according to an instruction received by the reception device 70 or the like.

In step ST210, the washer operation rate reference value is a value based on a ratio of the washer standard operation time with respect to time including the washer standard operation time calculated in step ST208 and the shortest standby time. Examples of the washer operation rate reference value include a value obtained by multiplying the ratio of the washer standard operation time with respect to the sum of the washer standard operation time calculated in step ST208 and the shortest standby time, by an adjustment factor. Examples of the adjustment factor according to the present embodiment include 0.9. The adjustment factor of “0.9” is merely an example, and the adjustment factor may be another decimal number less than 1. Further, the adjustment factor may be a fixed value or a variable value.

Further, although an example of the embodiment has been described in which the ratio of the washer standard operation time with respect to the sum of the washer standard operation time and the shortest standby time is multiplied by the adjustment factor, this is merely an example, and a value which is obtained by adding or subtracting an adjustment value to the ratio of the washer standard operation time with respect to the sum of the washer standard operation time and the shortest standby time may be used as the washer operation rate reference value.

In step ST212, the derivation unit 98E calculates an operation rate of each washer 52 for each examination time slot on the peak operation date specified in step ST206 with reference to the washer management database 112. After the process in step ST212 is executed, the medical service support process proceeds to step ST213.

The operation rate of the washer 52 is calculated by the derivation unit 98E according to the expression “(operation time of washer 52)/{(operation time of washer 52)+(standby time)}” for each examination time slot on the peak operation date. The “standby time” used in the denominator of this expression refers to washer standby time shown in FIG. 2, that is, the time from when washing of the endoscope 24 by the washer 52 is ended (for example, from when the process of step ST32 shown in FIG. 2 is ended) to when washing of the endoscope 24 by the washer 52 is started (for example, to when the process of step ST28 shown in FIG. 2 is started again). Further, the “standby time” used in the denominator of this expression is calculated by the derivation unit 98E based on the washing information 66 stored in the washer management database 112.

In step ST213, the specifying unit 98G specifies the examination time slot in which the operation status of the washer 52 peaks on the peak operation date (hereinafter, also referred to as a “peak operation time slot”) with reference to the washer management database 112 (see FIG. 9). After the process in step ST213 is executed, the medical service support process proceeds to step ST214.

The peak operation time slot refers to an examination time slot in which the operation statuses of all the washers 52 peak on the peak operation date. Hereinafter, for convenience of explanation, the peak operation date and the peak operation time slot are also referred to as “peak operation date and time”. The peak operation date and time is an example of “a time period when the operation degree of the washer is equal to or higher than the reference degree” according to the present disclosed technology. The operation degree of the medical device indicates information that indicates a ratio of operation of the medical device that can be acquired by the medical service support device and is indicated by, for example, a value based on the number of endoscopies. The value based on the number of endoscopies indicates, for example, the number of endoscopies for each predetermined period such as for each day or for each hour. Further, the value based on the number of endoscopies indicates a total number of endoscopies, which are executed by the plurality of medical devices, for each predetermined period in a case where there are a plurality of medical devices that can be acquired by the medical service support device. The value based on the number of endoscopies may be obtained from the information of the examination start time point and the examination end time point of each endoscopy with reference to the endoscopy management database. The reference degree is a reference value. More specifically, it is the reference value for determining a period during which endoscopy is intensively performed, such as the peak operation date or the peak operation time. The reference value may be a fixed value set in advance, or a variable value changed according to a user input. Further, the reference value may be a variation value calculated based on the number of endoscopies performed in the past. The reference value may be, for example, an average value of the number of endoscopies performed in the past or may be the maximum value of the number of endoscopies performed in the past or a value obtained by adding or multiplying the maximum value by a predetermined value. Further, in a case where the operation degree of the medical device indicates the number of endoscopies for each first period (for example, one day), the highest value, which is within the number of endoscopies for each first period in a second period (for example, one week) longer than the first period, may be defined as the reference value.

Further, here, the examination time slot in which an average value of the operation time of all the washers 52 on the peak operation date is the maximum is defined as the peak operation time slot. However, this is merely an example, and the examination time slot in which the average value of the operation rates calculated in step ST212 is the maximum may be defined as the peak operation time slot, the examination time slot in which the quantity of the washers 52 which are in operation is the highest may be used, and the examination time slot in which the median value of the operation time of the washer 52 is the maximum may be used. As described above, the examination time slot, which is selected from a value based on the quantity of the washers 52 and/or on the statistical value of the operation time, may be sufficient.

The peak operation date and time obtained in this manner is an example of “a time period when a value based on the number of times the washer is used is equal to or greater than the reference value” according to the present disclosed technology. Further, although the peak operation date and the peak operation date and time are exemplified here, this is merely an example, and a date or a date and time designated by the user or the like may be used instead of the peak operation date and the peak operation date and time. Further, although the peak operation date and time is exemplified here, this is merely an example, and the peak operation date may be applied instead of the peak operation date and time.

In step ST214, the acquisition unit 98C acquires, from the support device management database 113, the washer ID that can specify the washer 52 of which the operation rate calculated in step ST212 is within the operation rate range defined based on the washer operation rate reference value calculated in step ST210. After the process in step ST214 is executed, the medical service support process proceeds to step ST216.

In step ST214, the operation rate range, which is defined based on the washer operation rate reference value, refers to a range in which the operation rate of the washer 52 is equal to or greater than the washer operation rate reference value. That is, the washer operation rate reference value is an operation rate of an upper limit guideline to be compared with the operation rate of the washer 52. Therefore, in step ST214, the washer 52 having an operation rate equal to or greater than the upper limit guideline is specified by the acquisition unit 98C.

In step ST216, the derivation unit 98E calculates the washing wait time (see FIG. 2) for each washer 52 in the peak operation date and time specified in step ST206, with reference to the support device management database 113. After the process in step ST216 is executed, the medical service support process proceeds to step ST218 shown in FIG. 11B.

In step ST216, the washing wait time for each washer 52 is the time from the examination end time point to the washing start time point related to the endoscope 24 to be washed by the washer 52. The examination end time point is specified by using the endoscopy management database 110, and the washing start time point is specified by using the washer management database 112.

In step ST218 shown in FIG. 11B, the generation unit 98F generates first specifiable information that can specify a relationship between the washing wait time calculated in step ST216 and the washer 52 specified based on the washer ID acquired in step ST214. After the process in step ST218 is executed, the medical service support process proceeds to step ST220.

In step ST218, the first specifiable information is, for example, information that includes information in which the washing wait time calculated in step ST216 and the washer ID acquired in step ST214 are associated with each other for each washer ID acquired in step ST214.

In step ST220, the generation unit 98F generates first difference degree information that indicates a first difference degree which is a difference degree between the longest washing wait time and first shortest washing wait time. Here, the first shortest washing wait time is an example of “reference washing wait time” according to the present disclosed technology. After the process in step ST220 is executed, the medical service support process proceeds to step ST222.

In step ST220, the longest washing wait time is the longest time of the washing wait time for each washer ID included in the first specifiable information generated in step ST218. The first shortest washing wait time is time selected as the shortest time required from when the endoscopy is ended to when washing of the endoscope 24, which is used in an endoscopy, by the washer 52 is started. In the present step ST220, as an example of the first shortest washing wait time, the shortest time of the washing wait time for each washer ID included in the first specifiable information generated in step ST218 is used.

The first difference degree is, for example, a value obtained by subtracting the first shortest washing wait time from the longest washing wait time. However, this is merely an example, and the first difference degree may be a value indicating a deviation degree between the longest washing wait time and the first shortest washing wait time, such as a ratio of the first shortest washing wait time with respect to the longest washing wait time.

In step ST222, the generation unit 98F determines whether or not the first difference degree, which is indicated by the first difference degree information generated in step ST220, exceeds a first difference degree threshold value. Examples of the first difference degree threshold value include a value indicating a difference degree corresponding to “(predetermined time as longest washing wait time)/(predetermined time as first shortest washing wait time)=1.5”. Note that “1.5”, which is exemplified here, is merely an example, and other values may be used. Further, the first difference degree threshold value may be a fixed value or may be a variable value that is changed according to an instruction, which is provided by the user or the like, and/or at least one condition that is changed depending on a status.

In step ST222, in a case where the first difference degree indicated by the first difference degree information generated in step ST220 does not exceed the first difference degree threshold value, a negative determination is made, and the medical service support process proceeds to step ST226. In step ST222, in a case where the first difference degree indicated by the first difference degree information generated in step ST220 exceeds the first difference degree threshold value, a positive determination is made, and the medical service support process proceeds to step ST224.

In step ST224, the generation unit 98F generates first notification information for notifying that the manual washing has a possibility of overloading. The overloading of the manual washing means, for example, that there is a high probability that the manual washing staff is insufficient, and/or that there is a high probability that washing wait occurs for the washer 52 (particularly, dual-endoscope washer).

The first notification information also includes the first difference degree information. The information based on the first notification information is visualized and displayed in the medical service support screen 114 (see FIG. 12).

In step ST226, the generation unit 98F generates second specifiable information that can specify a relationship between the washing wait time and the examination time slot. After the process in step ST226 is executed, the medical service support process proceeds to step ST228.

In step ST226, the generation unit 98F calculates the washing wait time for each examination time slot and for each washer 52 for the peak operation date specified in step ST206 with reference to the support device management database 113. The generation unit 98F generates information which includes information in which the examination time slot of the peak operation date specified in step ST206 and the calculated washing wait time are associated with each other, for each washer 52, as the second specifiable information.

In step ST228, the generation unit 98F generates second difference degree information that indicates a second difference degree which is a difference degree between peak time slot washing wait time and second shortest washing wait time. Here, the second shortest washing wait time is an example of “reference washing wait time” according to the present disclosed technology. After the process in step ST228 is executed, the medical service support process proceeds to step ST230.

The peak time slot washing wait time refers to average time of all the washing wait times associated with all the washer IDs included in the peak operation time slot specified in step ST206 of all the washing wait times included in the second specifiable information generated in step ST226. For example, in a case where the peak operation time slot is around 11:00 on February 10, 20XX (Monday) and the second specifiable information includes information in which the washing wait time and the examination time slot are associated with each other for each of the first to fifth washers as the plurality of washers 52, the average time of the washing wait times around 11:00 on February 10, 20XX (Monday) for each of the first to fifth washers included in the second specifiable information is used as the peak time slot washing wait time.

The second shortest washing wait time is time selected as the shortest time required from when the endoscopy is ended to when washing of the endoscope 24, which is used in an endoscopy, by the washer 52 is started. In the present step ST228, as an example of the second shortest washing wait time, the shortest time is used of all the washing wait times associated with all the washer IDs included in the peak operation time slot specified in step ST206 of all the washing wait times included in the second specifiable information generated in step ST226.

The second difference degree is, for example, a value obtained by subtracting the second shortest washing wait time from the peak time slot washing wait time. However, this is merely an example, and the second difference degree may be a value indicating a deviation degree between the peak time slot washing wait time and the second shortest washing wait time, such as a ratio of the second shortest washing wait time with respect to the peak time slot washing wait time.

In step ST230, the generation unit 98F determines whether or not the second difference degree, which is indicated by the second difference degree information generated in step ST228, exceeds a second difference degree threshold value. Examples of the second difference degree threshold value include a value indicating a difference degree corresponding to “(predetermined time as peak time slot washing wait time)/(predetermined time as second shortest washing wait time)=1.5”. Note that “1.5”, which is exemplified here, is merely an example, and other values may be used. Further, the second difference degree threshold value may be a fixed value or may be a variable value that is changed according to an instruction, which is provided by the user or the like, and/or at least one condition that is changed depending on a status.

In step ST230, in a case where the second difference degree indicated by the second difference degree information generated in step ST228 does not exceed the second difference degree threshold value, a negative determination is made, and the medical service support process proceeds to step ST234 shown in FIG. 11C. In step ST230, in a case where the second difference degree indicated by the second difference degree information generated in step ST228 exceeds the second difference degree threshold value, a positive determination is made, and the medical service support process proceeds to step ST232.

In step ST232, the generation unit 98F generates second notification information for notifying that the manual washing has a possibility of overloading. The second notification information also includes the second difference degree information. The information based on the second notification information is visualized and displayed in the medical service support screen 116 (see FIG. 13).

In step ST234 shown in FIG. 11C, the derivation unit 98E refers to the washer management database 112 to determine whether or not the dual-endoscope washer is used at each of the peak operation dates and the peak operation dates and times specified in step ST206. Here, “dual-endoscope washer is used” is determined by the derivation unit 98E as to whether or not the dual-endoscope washer is operated regardless of whether or not the two endoscopes 24 are washed simultaneously by one dual-endoscope washer.

In step ST234, in a case where the dual-endoscope washer is not used, a negative determination is made, and the medical service support process proceeds to step ST242. In step ST234, in a case where the dual-endoscope washer is used, a positive determination is made, and the medical service support process proceeds to step ST236. Further, in a case where a negative determination is made in the present step ST238, that is, in a case where the dual-endoscope washer is used, it is an example of “a case where endoscopes are washed by a washer in a unit of a plurality of endoscopes” according to the present disclosed technology.

In step ST236, the derivation unit 98E calculates the difference degree between the washing wait times (hereinafter, also referred to as a “washing wait times difference degree”) classified by the default unit. After the process in step ST236 is executed, the medical service support process proceeds to step ST238.

Here, the washing wait times difference degree is, for example, an absolute value of a difference between the washing wait times. However, this is merely an example, and any value may be used as long as it indicates the deviation degree between one washing wait time and the other washing wait time, such as a ratio of the other washing wait time with respect to the one washing wait time.

The washing wait times difference degree is categorized as a first washing wait times difference degree, which is a difference degree between the washing wait times classified by the washer ID as the default unit and a second washing wait times difference degree, which is a difference degree between the washing wait times classified by the examination time slot as the default unit.

The first washing wait times difference degree is a difference degree between the washing wait times classified by the washer ID, for example, at the peak operation date and time (for example, around 11:00 on February 10, 20XX (Monday)). In this case, the derivation unit 98E calculates the first washing wait times difference degree with reference to the first specifiable information generated in step ST218.

Here, although the washer ID is exemplified as the default unit, the present embodiment is not limited to this, and as described above, the related information that is related to the washer 52 may be used as the default unit, such as the information capable of specifying that the washer 52 is a single-endoscope washer, the information capable of specifying that the washer 52 is a dual-endoscope washer, the information capable of specifying which sales series the washer 52 belongs to, or the information capable of specifying the manufacturer of the washer 52.

The second washing wait times difference degree is a difference degree between the washing wait times classified by the examination time slot, for example, at the peak operation date (for example, on February 10, 20XX (Monday)). Here, the washing wait times difference degree that is classified for each examination time slot refers to, for example, a difference degree between an average value of the washing wait times for all the washers 52 in one examination time slot of the two examination time slots to be compared and an average value of the washing wait times for all the washers 52 in the other examination time slot. In this case, the derivation unit 98E calculates the second washing wait times difference degree with reference to the second specifiable information generated in step ST226.

In step ST238, the generation unit 98F determines whether or not the washing wait times difference degree, which is generated in step ST236, exceeds a third difference degree threshold value. Examples of the third difference degree threshold value include the same value as the first difference degree threshold value or the second difference degree threshold value. The third difference degree threshold value may be a fixed value or may be a variable value that is changed according to an instruction, which is provided by the user or the like, and/or at least one condition that is changed depending on a status.

In step ST238, in a case where the washing wait times difference degree generated in step ST236 does not exceed the third difference degree threshold value, a negative determination is made, and the medical service support process proceeds to step ST242. In step ST238, in a case where the washing wait times difference degree generated in step ST236 exceeds the third difference degree threshold value, a positive determination is made, and the medical service support process proceeds to step ST240.

In step ST240, the generation unit 98F generates causal relationship information that indicates a causal relationship between a washing wait times difference degree calculated in step ST236 and a fact that the endoscope 24 is washed by the dual-endoscope washer. After the process in step ST240 is executed, the medical service support process proceeds to step ST242.

Here, the causal relationship refers to, for example, a relationship between the washing wait times difference degree and the fact that the endoscope 24 is washed by the dual-endoscope washer, and that washing of the endoscope 24 may be delayed due to the washing of the endoscope 24 by the dual-endoscope washer. The causal relationship information, which is generated by the generation unit 98F, is visualized and displayed on the medical service support screen 114 (see FIG. 12) and on the medical service support screen 116 (see FIG. 13).

In step ST242, the generation unit 98F generates the medical service support information according to various types of information. After the process in step ST242 is executed, the medical service support process proceeds to step ST244.

In step ST242, the various types of information refer to the peak operation date, the peak operation date and time, the first specifiable information, the second specifiable information, the first notification information, the second notification information, the causal relationship information, and the like. The medical service support information includes the peak operation date, the peak operation date and time, the first specifiable information, and the second specifiable information. Further, the medical service support information selectively includes the first notification information, the second notification information, and the causal relationship information. That is, in a case where the first notification information is generated in step ST224, the first notification information is included in the medical service support information, in a case where the second notification information is generated in step ST232, the second notification information is included in the medical service support information, and in a case where the causal relationship information is generated in step ST240, the causal relationship information is included in the medical service support information.

The medical service support information is information that is capable of being represented via a comment, a figure, or the like by a presentation device (here, the display 72 as an example) and includes screen information indicating the medical service support screen 114 (see FIG. 12) and the medical service support screen 116 (see FIG. 13). Here, although the screen information is generated by the medical service support device 20 as an example of the embodiment, this is merely an example, and the screen information may be generated by another device such as the endoscope part management device 12 or the like, for example.

As described in detail later, on the medical service support screen 114, a graph, a comment, and the like that visualize information in which the washing wait time of the peak operation date and time and the washer ID are associated with each other are included as the medical service support information. Further, on the medical service support screen 116, a graph, a comment, and the like that visualize information in which the washing wait time of the peak operation date and the examination time slot are associated with each other are included as the medical service support information.

In step ST244, the transmission unit 98B transmits the medical service support information, which is generated in step ST242, to the endoscope part management device main body 68 via the communication I/F 104 (see FIG. 7). After the process in step ST244 is executed, the medical service support process proceeds to step ST246.

In step ST246, the transmission unit 98B determines whether or not a condition for ending the medical service support process (hereinafter, referred to as a “medical service support process end condition”) is satisfied. Examples of the medical service support process end condition include a condition that an instruction for ending the medical service support process is received by the reception device (not shown).

In step ST246, in a case where the medical service support process end condition is not satisfied, a negative determination is made, and the medical service support process proceeds to step ST200 shown in FIG. 11A. In step ST246, in a case where the medical service support process end condition is satisfied, a positive determination is made, and the medical service support process is ended.

In a case where the medical service support information is transmitted by executing the process of step ST244, as described above, the medical service support information is received through the support device communication I/F 84 of the endoscope part management device 12 (see step ST110 shown in FIG. 10). As shown in step ST112 shown in FIG. 10, the display control unit 78D of the endoscope part management device 12 generates the medical service support screen 114 (see FIG. 12) and the medical service support screen 116 (see FIG. 13) based on the medical service support information, and the generated medical service support screen 114 and medical service support screen 116 are displayed on the display 72 (FIG. 1 and FIG. 3) (see FIG. 12 and FIG. 13).

As an example, as shown in FIG. 12, on the medical service support screen 114, a screen title display field 114A, a washing wait time calculation target date display field 114B, an examination support graph 114C, an examination support comment display field 114D, and a guidance comment display field 114E are displayed.

In the screen title display field 114A, a title which can enable recognition that the medical service support screen 114 is a screen for representing variation in the washing wait time, that is, the manual washing time for each washer 52, is displayed. In the example shown in FIG. 12, “variation in washing wait time (manual washing time) for each washer” is displayed in the screen title display field 114A. Since the washing wait time of the washer 52 can be regarded as the manual washing time, for example, a title such as “variation in manual washing time for each washer” or “variation in manual washing time” may be displayed in the screen title display field 114A.

In the washing wait time calculation target date display field 114B, the peak operation date and time, which is specified in step ST206 shown in FIG. 11A, is displayed. In the example shown in FIG. 12, the peak operation date and time is represented by the year, month, date, day of the week, and time slot, such as “target date: around 11:00 on 2/10/20XX (Monday)”.

The examination support graph 114C is an example of a “figure” according to the present disclosed technology. The examination support graph 114C is a graph created by the display control unit 78D based on the first specifiable information, the first notification information, and the like, and shows the washing wait time for each washer 52 at the peak operation date and time. A horizontal axis of the examination support graph 114C is the washer ID that can specify each washer 52, and a vertical axis of the examination support graph 114C is the washing wait time. In the example shown in FIG. 12, although a bar graph is illustrated, the present disclosed technology is not limited to this, and other graphs such as a line graph, a pie chart, and a band graph may be used, and figures other than graphs such as a bubble chart may be used.

In the example shown in FIGS. 12, A-1, A-2, A-3, A-4, and A-5 are arranged on the horizontal axis as the washer IDs, and among these washers 52, the washing wait time of the washer 52 of A-4 is the shortest, and the washing wait time of the washer 52 of A-3 is the longest. The washing wait time of the washer 52 of A-3 is 1.5 times the washing wait time of the washer 52 of A-4. In the examination support graph 114C, a background 114C1 that is from 6.6 minutes, which is the washing wait time of the washer 52 of A-4, to 10 minutes, which is the washing wait time of the washer 52 of A-3, is displayed in a manner distinguishable (in the example shown in FIG. 12, diagonal line-shaped hatching) from other regions. Further, the text “1.5 times” is assigned with respect to a range of the washing wait time from 6.6 minutes to 10 minutes in the background 114C1, and the first difference degree is represented by the examination support graph 114C in a manner in which the level of the difference degree is visualized.

In the examination support comment display field 114D, a comment contributing to the elimination of the variation in the washing wait time, that is, the manual washing time for each washer 52, is displayed. The comment in the examination support comment display field 114D is a comment created by the display control unit 78D based on the peak operation date and time, the first notification information, the causal relationship information, and the like.

In the examination support comment display field 114D, a comment (in the example shown in FIG. 12, a comment such as “Shows washing wait time (manual washing time) around 11:00 where the operation rate of the washer is the highest”) announcing that the washing wait time, that is, the manual washing time at the peak operation date and time, is shown in the examination support graph 114C is displayed.

Further, in the examination support comment display field 114D, a comment (in the example shown in FIG. 12, a comment such as “There is a difference of 1.5 times between the shortest washing wait time and the longest washing wait time”) indicating the first difference degree (see step ST220 in FIG. 11B) shown in step ST220 shown in FIG. 11B, that is, a comment indicating how much the longest washing wait time differs from the first shortest washing wait time, is displayed, as a comment for notifying that the manual washing has a possibility of overloading.

Further, in the examination support comment display field 114D, a comment (hereinafter, also referred to as a “first alert comment”) such as “Please check whether the dual-endoscope washer is in a washing wait state” is displayed, as information in which the causal relationship information generated in step ST240 is visualized.

The first alert comment may be displayed in a case where a specific condition is satisfied even in a case where the causal relationship information is not generated in step ST240 shown in FIG. 11C. For example, the specific condition refers to, for example, a condition that the first difference degree exceeds the first difference degree threshold value (in a case where a positive determination is made in step ST222 shown in FIG. 11B).

Further, for example, the first alert comment may be a comment such as “The dual-endoscope washer is being used around 11:00 on 2/11/20XX (Monday). Please check whether the dual-endoscope washer is in a washing wait state.” that clarifies the peak operation date and time and the fact that the dual-endoscope washer is used.

Note that in the example shown in FIG. 12, by displaying the comment “There is a difference of 1.5 times between the shortest washing wait time and the longest washing wait time” in the examination support comment display field 114D, notification that manual washing has a possibility of overloading is sent, but this is merely an example, and instead of or together with this comment, comments such as “Manual washing has a possibility of overloading” and/or “Manual washing staff may be insufficient” may be displayed in the examination support comment display field 114D.

In the guidance comment display field 114E, a guidance comment is displayed. In the example shown in FIG. 12, in the guidance comment display field 114E, as a comment indicating guidance to the medical service support screen 116 (see FIG. 13), a comment which indicates that the washing wait time for each examination time slot can be checked on the next page is displayed.

As an example, as shown in FIG. 13, on the medical service support screen 116, a screen title display field 116A, a washing wait time calculation target date display field 116B, an examination support graph 116C, an examination support comment display field 116D, and a guidance comment display field 116E are displayed.

In the screen title display field 116A, a title which can enable recognition that the medical service support screen 116 is a screen for representing variation in the washing wait time, that is, the manual washing time for each examination time slot, is displayed. In the example shown in FIG. 13, “variation in washing wait time (manual washing time) for each examination time slot” is displayed in the screen title display field 116A. Since the washing wait time of the washer 52 can be regarded as the manual washing time, for example, a title such as “variation in manual washing time for each examination time slot” or “variation in manual washing time” may be displayed in the screen title display field 116A.

In the washing wait time calculation target date display field 116B, the peak operation date, which is specified in step ST206 shown in FIG. 11A, is displayed. In the example shown in FIG. 13, the peak operation date is represented by the year, month, date, and day of the week, such as “target date: 2/10/20XX (Monday)”.

The examination support graph 116C is an example of a “figure” according to the present disclosed technology. The examination support graph 116C is a graph created by the display control unit 78D based on the second specifiable information, the second notification information, and the like, and shows the washing wait time for each examination time slot at the peak operation date. A horizontal axis of the examination support graph 116C is the examination time slot, and a vertical axis of the examination support graph 116C is the washing wait time. In the example shown in FIG. 12, although a bar graph is illustrated, the present disclosed technology is not limited to this, and other graphs such as a line graph, a pie chart, and a band graph may be used, and figures other than graphs such as a bubble chart may be used.

In the example shown in FIG. 13, around 9:00, 10:00, 11:00, 12:00, 13:00, 14:00, 15:00, and 16:00 are arranged on the horizontal axis as the examination time slots. Further, the washing wait time is represented by a bar graph for each examination time slot. The washing wait time corresponding to the examination time slot is the average time of the washing wait times of all the washers 52 in the examination time slot.

Further, in the examination support graph 116C, a background 116C1 of the peak operation date and time (in the example shown in FIG. 13, around 11:00) is displayed in a manner distinguishable (in the example shown in FIG. 12, diagonal line-shaped hatching) from other regions.

Further, in the examination support graph 116C, the washing wait time around 9:00 is the shortest among all the examination time slots around 9:00 to around 16:00. Further, the washing wait time around 11:00 on the peak operation date and time is 2.0 times the washing wait time around 9:00. In the examination support graph 116C, a background 116C2 that is from 10 minutes, which is the washing wait time around 9:00, to 20 minutes, which is the washing wait time around 11:00, is displayed in a manner distinguishable (in the example shown in FIG. 13, dot-shaped hatching) from other regions. Further, the text “2.0 times” is assigned with respect to a range from 10 minutes, which is the washing wait time around 9:00, to 20 minutes, which is the washing wait time around 11:00, in the background 116C2, and the second difference degree is represented by the examination support graph 116C in a manner in which the level of the difference degree is visualized.

In the examination support comment display field 116D, a comment contributing to the elimination of the variation in the washing wait time, that is, the manual washing time for each washer 52 between examination time slots on the peak operation date, is displayed. The comment in the examination support comment display field 116D is a comment created by the display control unit 78D based on the peak operation date and time, the second notification information, the causal relationship information, and the like.

In the examination support comment display field 116D, a comment (in the example shown in FIG. 13, a comment such as “Shows the transition in washing wait time on the peak date of endoscopy (2/10/20XX (Monday))”) announcing that the change of the washing wait time, that is, the manual washing time at the peak operation date, is shown in the examination support graph 116C is displayed.

Further, in the examination support comment display field 116D, a comment indicating the second difference degree shown in step ST228 shown in FIG. 11B, that is, a comment (in the example shown in FIG. 13, a comment such as “There is a difference of 2.0 times between the washing wait time and the shortest washing wait time at the peak time slot (around 11:00) of endoscopy”) indicating how much the peak time slot washing wait time differs from the second shortest washing wait time is displayed, as a comment for notifying that the manual washing has a possibility of overloading.

Further, in the examination support comment display field 116D, a comment (hereinafter, also referred to as a “second alert comment”) such as “Please check whether the dual-endoscope washer is in a washing wait state at the peak time slot (around 11:00)” is displayed, as information in which the causal relationship information generated in step ST240 is visualized.

The second alert comment may be displayed in a case where a specific condition is satisfied even in a case where the causal relationship information is not generated in step ST240 shown in FIG. 11C. For example, the specific condition refers to, for example, a condition that the second difference degree exceeds the second difference degree threshold value (in a case where a positive determination is made in step ST230 shown in FIG. 11B).

Note that in the example shown in FIG. 13, by displaying the comment “There is a difference of 2.0 times between the washing wait time and the shortest washing wait time at the peak time slot (around 11:00) of endoscopy” in the examination support comment display field 116D, notification that the manual washing has a possibility of overloading is sent, but this is merely an example, and instead of or together with this comment, comments such as “Manual washing has a possibility of overloading” and/or “Manual washing staff may be insufficient” may be displayed in the examination support comment display field 116D.

In the guidance comment display field 116E, a guidance comment is displayed. In the example shown in FIG. 13, in the guidance comment display field 116E, as a comment indicating guidance to the medical service support screen 114 (see FIG. 12), a comment which indicates that the washing wait time for each washer 52 can be checked on the previous page is displayed.

As described above, in the medical service support device 20 according to the present embodiment, the washing wait time from when the endoscopy is ended to when washing of the endoscope 24 by the washer 52 is started is acquired for each washer 52 (see step ST218 shown in FIG. 11B). In the medical service support device 20, the medical service support information in which the washing wait time and the washer 52 are associated with each other is transmitted to the endoscope part management device 12 (see step ST244 shown in FIG. 11C).

Further, in the medical service support device 20, the washing wait time from when the endoscopy is ended to when washing of the endoscope 24 by the washer 52 is started is acquired for each examination time slot (see step ST226 shown in FIG. 11B). In the medical service support device 20, the medical service support information in which the washing wait time and the examination time slot are associated with each other is transmitted to the endoscope part management device 12 (see step ST244 shown in FIG. 11C).

In the endoscope part management device 12, the medical service support screens 114 and 116 are generated based on the medical service support information and are displayed on the display 72 (see step ST112 shown in FIG. 10). Since the medical service support information is presented on the medical service support screens 114 and 116 in a visualized state to the user or the like, the user or the like can ascertain how tight the endoscope handling service is by comparing the washing wait times between the washers 52 and comparing the washing wait times between the examination time slots. Therefore, according to the present configuration, it is possible to support the efficient performance of the endoscope handling service.

Further, in the medical service support device 20, the washing wait time on the peak operation date and time is acquired for each washer 52. Further, in the endoscope part management device 12, the washing wait time for each washer 52 on the peak operation date and time is displayed on the medical service support screen 114. Therefore, according to the present configuration, the user or the like can ascertain the washing wait time for each washer on the peak operation date and time.

Further, in the medical service support device 20, the washing wait time on the peak operation date is acquired for each examination time slot. Further, in the endoscope part management device 12, the washing wait time for each examination time slot on the peak operation date is displayed on the medical service support screen 114. Therefore, according to the present configuration, the user or the like can ascertain the washing wait time for each examination time slot on the peak operation date.

Further, the peak operation date is a date on which the operation statuses of all the information processing apparatuses 36 peak, and the peak operation time slot is an examination time slot where the operation statuses of all the washers 52 peak. Therefore, according to the present configuration, the user or the like can ascertain the washing wait time at the examination time slot where the operation statuses of all the washers 52 peak, on the date on which the operation statuses of all the information processing apparatuses 36 peak.

Further, the peak operation date is a date on which an endoscopy is performed the highest number of times. Therefore, according to the present configuration, as compared with a case in which the medical service support information includes only the washing wait time, the user or the like can accurately ascertain the washing wait time on a date on which the washer 52 is used the highest number of times.

Further, in the medical service support device 20, information which includes information (see step ST218 shown in FIG. 11B) capable of specifying the relationship between the washing wait time and the washer 52 is generated as the medical service support information (see step ST242 shown in FIG. 11C). Therefore, according to the present configuration, it is possible to easily realize comparable presentation of the waiting washing wait times among the washers 52, as compared with a case where the medical service support information is information that cannot specify the relationship between the washing wait time and the washer 52, that is, the relationship between the washing wait time and the washer ID.

Further, in the medical service support device 20, information which includes information (see step ST226 shown in FIG. 11B) capable of specifying the relationship between the washing wait time and the examination time slot is generated as the medical service support information (see step ST242 shown in FIG. 11C). Therefore, according to the present configuration, it is possible to easily realize comparable presentation of the waiting washing wait times among the examination time slots, as compared with a case where the medical service support information is information that cannot specify the relationship between the washing wait time and the examination time slot.

Further, in the medical service support device 20, information which includes the first difference degree information (see step ST220 shown in FIG. 11B) indicating the first difference degree between the longest washing wait time and the first shortest washing wait time is generated as the medical service support information (see step ST242 shown in FIG. 11C). Therefore, according to the present configuration, the user or the like can ascertain how much the longest washing wait time differs from the first shortest washing wait time.

Further, in the medical service support device 20, information which includes the second difference degree information (see step ST228 shown in FIG. 11B) indicating the second difference degree between the peak time slot washing wait time and the second shortest washing wait time is generated as the medical service support information (see step ST242 shown in FIG. 11C). Therefore, according to the present configuration, the user or the like can ascertain how much the peak time slot washing wait time differs from the second shortest washing wait time.

Further, in the medical service support device 20, in a case where the first difference degree, which is indicated by the first difference degree information, exceeds the first difference degree threshold value (in a case where a positive determination is made in step ST222 shown in FIG. 11B), information which includes first notification information (see step ST224 shown in FIG. 11B) for notifying that the manual washing has a possibility of overloading is generated as the medical service support information (see step ST242 shown in FIG. 11C). Further, in a case where the second difference degree, which is indicated by the second difference degree information, exceeds the second difference degree threshold value (in a case where a positive determination is made in step ST230 shown in FIG. 11B), information which includes second notification information (see step ST232 shown in FIG. 11B) for notifying that the manual washing has a possibility of overloading is generated as the medical service support information (see step ST242 shown in FIG. 11C). Therefore, according to the present configuration, the user or the like can ascertain that the manual washing has a possibility of overloading.

Further, in the medical service support device 20, in a case where the dual-endoscope washer is used, information which includes the causal relationship information (see step ST240 shown in FIG. 11C) indicating the causal relationship between the washing wait times difference degree and the fact that the dual-endoscope washer is used is generated as the medical service support information (see step ST242 shown in FIG. 11C). Therefore, according to the present configuration, it is possible to suggest to the user or the like that the cause of the washing wait of the washer 52 may be due to the use of the dual-endoscope washer.

Further, the medical service support information is information that is capable of being represented via a comment and a figure (for example, the examination support graphs 114C and 116C) on the display 72. Therefore, according to the present configuration, it is possible for the user or the like to visually ascertain the medical service support information.

In the above-described embodiment, although the first difference degree between the longest washing wait time and the first shortest washing wait time is exemplified, the present disclosed technology is not limited to this, and another reference washing wait time may be applied instead of the first shortest washing wait time. Examples of the reference washing wait time other than the first shortest washing wait time include a statistical value (an average value, a median value, the most frequent value, the maximum value, the minimum value, or the like) of washing wait times in the examination time slot specified by the user or the like, or a statistical value (an average value, a median value, the most frequent value, the maximum value, the minimum value, or the like) of the washing wait times of the washer 52 specified by the user or the like.

In the medical service support process (see FIG. 11A) according to the embodiment, although the peak operation date is specified by executing the process of step ST206, the present disclosed technology is not limited to this. For example, the specifying unit 98G may derive a date on which the operation status of the washer 52 peaks as the peak operation date with reference to the washer management database 112 (see FIG. 9). The peak operation date in this case indicates a date on which the operation status of all the washers 52 installed in the washer room 50 peaks within a period designated in advance (for example, a period designated by the user or the like). Examples of the peak operation date in this case include a date on which the washer 52 is used the highest number of times. The date on which the washer 52 is used can be specified based on the washing start time point or the washing end time point of the washing information 66 stored in the washer management database 112. Further, other examples of the peak operation date include a date on which the number of times the washer standby time (see FIG. 2) falls below a standby time threshold value (for example, 50 minutes) is equal to or greater than a reference number of times. In other words, the operation degree of the medical device may be indicated by a value based on the number of times the examination standby time falls below the standby time threshold value, and the reference degree may be indicated by the reference number of times. The number of times the examination standby time falls below the standby time threshold value indicates the number of times the standby time for each predetermined period, such as for each day or for each hour, falls below the standby time threshold value. Further, in a case where there are a plurality of medical devices that can be acquired by the medical service support device, the number of times the examination standby time falls below the standby time threshold value indicates the total number of times the examination standby time falls below the standby time threshold value in the plurality of medical devices. The standby time threshold value may correspond to a preparation period required from the endoscopy end to the next endoscopy start in a medical installation facility, may be a fixed value, or may be a variable value set or changed by the user or the like. Further, the standby time threshold value may be a variation value calculated based on the examination standby time of the endoscope procedure service performed in the past. For example, the average value of the examination standby time of the endoscope procedure service performed in the past, the minimum value of the examination standby time of the endoscope procedure service performed in the past, or a value obtained by adding or multiplying a predetermined value to the minimum value may be used. Further, the reference number of times corresponding to the reference degree is a default value. More specifically, it is a default value for determining a period during which endoscopy is intensively performed, such as the peak date or the peak time. The default value may be a fixed value set in advance, or a variable value changed according to a user input. Further, the default value may be a variation value calculated based on the number of times the examination standby time in the past falls below the standby time threshold value. For example, the default value may be an average value of the number of times the examination standby time in the past falls below the standby time threshold value or a variation value calculated based on the number of times the examination standby time in the past falls below the standby time threshold value, for example, the maximum value of the number of times the examination standby time in the past falls below the standby time threshold value or a value obtained by adding or multiplying a predetermined value to the maximum value. Further, in a case where the operation degree of the medical device indicates the number of times the examination standby time falls below the standby time threshold value for each first period (for example, one day), the highest value, which is within the number of times the examination standby time falls below the standby time threshold value for each first period in a second period (for example, one week) longer than the first period, may be set as a default value.

Further, for example, the peak operation date may be a date on which the number of times the examination standby time (see FIG. 2) falls below the standby time threshold value (for example, 3 minutes) is equal to or greater than the reference number of times. In this case, it is possible to specify the peak operation date even in a case where the number of times an endoscopy is performed cannot be ascertained. Note that the standby time threshold value and the reference number of times may be fixed values or variable values that are changed by the user or the like.

Further, for example, the specifying unit 98G may specify a date on which the operation status of the endoscope 24 peaks as the peak operation date with reference to the support device management database 113 (see FIG. 9). The peak operation date in this case indicates a date on which the operation status of the endoscope 24 peaks within a period designated in advance (for example, a period designated by the user or the like). Examples of the peak operation date in this case include a date on which the endoscope 24 is used the highest number of times. The date on which the endoscope 24 is used can be specified based on the examination start time point, the examination end time point, the washing start time point, or the washing end time point stored in the support device management database 113. Further, other examples of the peak operation date include a date on which the number of times the endoscope standby time falls below the standby time threshold value (for example, 50 minutes) is equal to or greater than the reference number of times. The endoscope standby time refers to time from when washing of the endoscope 24 by the washer 52 is ended to when an endoscopy is started.

In the above-described embodiment, although an example of the embodiment has been described in which the causal relationship information is generated on a condition that the dual-endoscope washer is used regardless of whether one endoscope 24 or two endoscopes 24 are washed by the dual-endoscope washer, the present disclosed technology is not limited to this. For example, the causal relationship information may be generated on a condition that the fact that the two endoscopes 24 are washed by the dual-endoscope washer is checked. The fact that the two endoscopes 24 are washed by the dual-endoscope washer is determined in a case where the CPU 98 specifies that the washing start time points for two endoscopes 24 used by the dual-endoscope washer, which is specified by the washer ID related to the dual-endoscope washer, are the same time points, by using the washer management database 112.

In the embodiment described above, although the peak operation date is exemplified, the present disclosed technology is not limited to this, and the peak operation time period may be specified in a unit of time other than day, such as peak operation month or peak operation year.

In the embodiment described above, although the case has been described where the medical service support information is the examination support graph 114C and the information in which the comment can be represented, the present disclosed technology is not limited to this, and the medical service support information may be information that can be presented in a table. That is, the medical service support information may be any information that can be represented by at least one of a figure, a comment, or a table.

In the above-described embodiment, although the plurality of washers 52 including at least one dual-endoscope washer have been exemplified, the present disclosed technology is not limited to this. A washer 52 that washes the endoscope 24 in a unit of a plurality of endoscopes of three or more may be used instead of or together with the dual-endoscope washer.

In the embodiment described above, although an example of the embodiment has been described in which the operation of the information processing apparatus 36 is started by turning on the examination start button 38A, and the operation of the information processing apparatus 36 is stopped by turning off the examination start button 38A, the present disclosed technology is not limited to this. The operation of the information processing apparatus 36 may be started on a condition that the barcode 42 is read by the barcode reader 40, and the operation of the information processing apparatus 36 may be stopped on a condition that the barcode 42 is read again by the barcode reader 40. The same applies to the start and stop of the operation of the washer 52.

An example has been described in which the start operation and the stop operation of the information processing apparatus 36 are executed by pressing down the examination start button 38A, but this is merely an example. The examination start button 38A is not limited to a physical button. The operation of the information processing apparatus 36 may be started or the operation of the information processing apparatus 36 may be stopped by detecting nearness of at least one softkey displayed on a touch panel, such as a touch panel display. Further, the start operation and the stop operation of the information processing apparatus 36 may be controlled by a voice input and/or a gesture input. Further, a noncontact human sensor may be provided in a default region of the examination room 30 or the information processing apparatus 36, and the operation of the information processing apparatus 36 may be started or the operation of the information processing apparatus 36 may be stopped by causing the noncontact human sensor to detect a human body. The same applies to the control of the start operation and the stop operation by using the washing start button 60A of the washer 52.

In the embodiment described above, although an example of the embodiment has been described in which the medical service support information is generated based on the washing wait time and on the default unit (for example, the washer ID and the examination time slot) for the endoscope part, the present disclosed technology is not limited to this, and the medical service support information may be generated based on the washing wait time and on the default unit (for example, the washer ID and the examination time slot) in a case where an endoscopy is performed in units of clinical departments (for example, internal medicine or surgery) or medical institutions (for example, clinic or general hospital).

In the embodiment described above, although the examination room 30 has been exemplified as an example, it does not necessarily have to be one room, and may be a space obtained by dividing one room by a curtain and/or a stand or the like.

In the above embodiment, although the endoscope processor device 32 and the information processing apparatus 36 are separate units, the present disclosed technology is not limited to this, and the endoscope processor device 32 and the information processing apparatus 36 may be integrated. In this case, for example, a device corresponding to the information processing apparatus 36 may be incorporated into the endoscope processor device 32, or a device corresponding to the endoscope processor device 32 may be incorporated into the information processing apparatus main body 38.

In the embodiment described above, although an example of the embodiment has been described in which the medical service support information is visualized and presented to the user or the like by displaying the medical service support screens 114 and 116 on the display 72, the present disclosed technology is not limited to this. For example, the medical service support information may be audibly output by a sound reproducing device, or the medical service support information may be printed on a recording medium (for example, paper) by a printer and output, instead of or together with the visual presentation by the display 72 described above.

In the embodiment described above, although the endoscope handling service including an endoscopy, which is an example of the “endoscope procedure service” according to the present disclosed technology, has been described, the present disclosed technology is not limited to this, and a medical device handling service including a medical device procedure service other than an endoscopy may be used. For example, the endoscope procedure service may be a service including endoscopic surgery and/or endoscopic treatment. Examples of endoscopic surgery include laparoscopic surgery and/or treatment, thoracoscopic surgery and/or treatment, cystoscopic surgery and/or treatment, choledoscopic surgery and/or treatment, spinal endoscopy and/or therapy, angioscopy surgery and/or treatment, and epidural endoscopic surgery and/or treatment.

In the embodiment described above, although the endoscope 24, the information processing apparatus 36, and the washer 52 are described as examples of the medical device, the present disclosed technology is not limited to this, and the medical device may be a medical device other than the endoscope 24, the information processing apparatus 36, and the washer 52. Examples of the medical device other than the endoscope 24, the information processing apparatus 36, and the washer 52 include a medical management device (for example, a device having a processor and a memory) corresponding to the information processing apparatus 36, a medical accessory device that is attachably and detachably connected to a medical management device in the same way that the endoscope 24 is attachably and detachably connected to the information processing apparatus 36 (for example, replaceable ultrasonic probes and/or replaceable therapeutic tools used in dental treatment, or the like), and a washer that washes the medical accessory device similar to the washer 52 that washes the endoscope 24.

In the embodiment described above, although the barcode 42 and the subject barcode are exemplified, another two-dimensional code such as a quick response (QR) code (registered trademark) may be used, or a noncontact storage medium using radio frequency identification (RFID) technology may be used, instead of at least one of the barcode 42 or the subject barcode. Further, in this case, the endoscope 24 or a device that can acquire information which is capable of specifying a subject may be applied by recognizing these two-dimensional codes and/or noncontact storage media instead of the barcode readers 40 and 64.

In the embodiment described above, although the medical service support device 20 is exemplified, a computer, which is used together with the endoscope part management device 12 on-premises, may be caused to execute the medical service support processing program 108 (see FIG. 8) instead of the medical service support device 20. Further, the management information storage device 76 may also be used on-premises together with the endoscope part management device 12.

Further, in the embodiment described above, although an example of the embodiment has been described in which the medical service support processing program 108 is stored in the storage 100, the medical service support processing program 108 may be stored in any portable storage medium such as an SSD or universal serial bus (USB) memory, or the medical service support processing program 108 may be stored in a non-temporary storage medium. The medical service support processing program 108 stored in the non-temporary storage medium is installed, for example, in the support device server 74 or the like.

Further, in the embodiment described above, although the support device server 74 including the CPU 98, the storage 100, and the memory 102 has been exemplified, the present disclosed technology is not limited to this, and a device including an application specific integrated circuit (ASIC), a field-programmable gate array (FPGA), and/or a programmable logic device (PLD) may be applied instead of the support device server 74 or together with the support device server 74.

As a hardware resource for executing the medical service support process described in the above embodiment, the following various processors can be used. Examples of the processor include a CPU, which is a general-purpose processor that functions as a hardware resource for executing the medical service support process by executing software, that is, a program. Further, examples of the processor include a dedicated electric circuit, which is a processor having a circuit configuration specially designed for executing specific processing such as an FPGA, a PLD, or an ASIC. Any processor has a memory built in or connected to it, and any processor uses the memory to execute a terminal side process.

The hardware resource for executing the medical service support process may be configured with one of these various processors or may be configured with a combination (for example, a combination of a plurality of FPGAs or a combination of a CPU and an FPGA) of two or more processors of the same type or different types. Further, the hardware resource for executing the medical service support process may be one processor.

As an example of a configuration with one processor, first, one processor is configured with a combination of one or more CPUs and software, and there is an embodiment in which this processor functions as a hardware resource for executing the medical service support process. Secondly, as typified by a system-on-a-chip (SoC), there is an embodiment in which a processor that implements the functions of the entire system including a plurality of hardware resources for executing the medical service support process with one IC chip is used. As described above, the medical service support process is implemented by using one or more of the above-mentioned various processors as a hardware resource.

Further, as the hardware-like structure of these various processors, more specifically, an electric circuit in which circuit elements such as semiconductor elements are combined can be used. Further, the above-mentioned terminal side process is merely an example. Therefore, it goes without saying that unnecessary steps may be deleted, new steps may be added, or the processing order may be changed within a range that does not deviate from the purpose.

The contents described above and the contents shown in the illustrations are detailed explanations of the parts related to the present disclosed technology and are merely an example of the present disclosed technology. For example, the description related to the configuration, function, action, and effect described above is an example related to the configuration, function, action, and effect of a portion according to the present disclosed technology. Therefore, it goes without saying that unnecessary parts may be deleted, new elements may be added, or replacements may be made to the contents described above and the contents shown in the illustrations, within the range that does not deviate from the purpose of the present disclosed technology. Further, in order to avoid complications and facilitate understanding of the parts of the present disclosed technology, in the contents described above and the contents shown in the illustrations, the descriptions related to common technical knowledge or the like that do not require special explanation in order to enable the implementation of the present disclosed technology are omitted.

In the present specification, “A and/or B” is synonymous with “at least one of A or B”. That is, “A and/or B” means that it may be only A, it may be only B, or it may be a combination of A and B. Further, in the present specification, in a case where three or more matters are connected and expressed by “and/or”, the same concept as “A and/or B” is applied.

All documents, patent applications, and technical standards described in the present specification are incorporated in the present specification by reference to the same extent in a case where it is specifically and individually described that the individual documents, the patent applications, and the technical standards are incorporated by reference.

Further, the following Supplementary Notes will be disclosed with respect to the above embodiments.

(Supplementary Note 1)

A medical service support device includes: a processor; and a memory that is connected to or built into the processor, in which the processor is configured to: acquire a washing wait time from when a medical device procedure service using a medical accessory device that is attachably and detachably connected to a medical management device is ended to when washing of the medical accessory device by a washer is started, for each default unit capable of classifying the washing wait time; and output medical service support information in which the washing wait time and the default unit are associated with each other.

According to the configuration described in Supplementary Note 1, it is possible to support the efficient performance of the medical device handling service.

(Supplementary Note 2)

In the medical service support device according to Supplementary Note 1, the default unit is related information that is related to the washer.

(Supplementary Note 3)

In the medical service support device according to Supplementary Note 1, the default unit is a time slot.

What is claimed is:

1. A medical service support device comprising:
a processor; and
a memory that is connected to or built into the processor,
wherein the processor is configured to:
acquire a washing wait time from when an endoscope procedure service using an endoscope is ended to when washing of the endoscope by a washer is started, for each default unit capable of classifying the washing wait time; and
output medical service support information in which the washing wait time and the default unit are associated with each other.

2. The medical service support device according to claim 1,
wherein a plurality of the washers are present, and
the processor is configured to acquire the washing wait time for each of the plurality of washers.

3. The medical service support device according to claim 1,
wherein the medical service support information is information that includes information capable of specifying a relationship between the washing wait time and the default unit.

4. The medical service support device according to claim 1,
wherein the medical service support information is information that includes difference degree information indicating a difference degree between the washing wait time and a reference washing wait time.

5. The medical service support device according to claim 4,
wherein the reference washing wait time is a time selected as a shortest time required from when the endoscope procedure service is ended to when the washing by the washer is started.

6. The medical service support device according to claim 4,
wherein the washing wait time includes a manual washing time during which manual washing is performed on the endoscope, and
in a case where the difference degree, which is indicated by the difference degree information, exceeds a difference degree threshold value, the medical service support information is information that includes notification information for notifying that the manual washing has a possibility of overloading.

7. The medical service support device according to claim 1,
wherein a plurality of the washers are present,
at least one of the plurality of washers is capable of washing the endoscope in a unit of a plurality of endoscopes, and
in a case where the endoscope is washed by the washer in the unit of a plurality of endoscopes, the medical service support information is information that includes causal relationship information indicating a causal relationship between a difference degree between the washing wait times, which are classified by the default unit, and a fact that the endoscope is washed by the washer in the unit of a plurality of endoscopes.

8. The medical service support device according to claim 1,
wherein the medical service support information is information capable of being represented via at least one of a comment, a diagram, or a table by a presentation device.

9. The medical service support device according to claim 1,
wherein the default unit is related information that is related to the washer.

10. The medical service support device according to claim 1,
wherein the default unit is a time slot.

11. The medical service support device according to claim 1,
wherein the processor is configured to acquire the washing wait time for each default unit for a time period when an operation degree of a medical device, which is used in an endoscope handling service including the endoscope procedure service, is equal to or higher than a reference degree.

12. The medical service support device according to claim 11,
wherein the time period when the operation degree of the medical device is equal to or higher than the reference degree is a time period when a value based on the number of endoscope procedure services is equal to or greater than a reference value.

13. The medical service support device according to claim 11,
wherein the time period when the operation degree of the medical device is equal to or higher than the reference degree is a time period when the number of times standby time of the endoscope procedure service falls below a standby time threshold value is equal to or greater than a reference number of times.

14. The medical service support device according to claim 11,
wherein, among an information processing apparatus, which is used together with the endoscope, and the washer, the medical device is at least the information processing apparatus.

15. A medical service support method comprising:
acquiring a washing wait time from when an endoscope procedure service using an endoscope is ended to when washing of the endoscope by a washer is started, for each default unit capable of classifying the washing wait time; and
outputting medical service support information in which the washing wait time and the default unit are associated with each other.

16. A non-transitory computer-readable storage medium storing a program executable by a computer to perform a process comprising:
acquiring a washing wait time from when an endoscope procedure service using an endoscope is ended to when washing of the endoscope by a washer is started, for each default unit capable of classifying the washing wait time; and
outputting medical service support information in which the washing wait time and the default unit are associated with each other.
